SEQUENCE LISTING (35 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 22 aa, Artificial Sequence
  synthetic construct; mutant insulin
GVNQHLCGSELVEALYLVCYER

SEQ ID NO: 4 - protein, 22 aa, Artificial Sequence
  synthetic construct; mutant insulin chain
FVNQHLCGSELVEALYLVCFER

SEQ ID NO: 5 - protein, 22 aa, Artificial Sequence
  synthetic construct; mutant insulin chain
FVNQHLCGSELVEALYLVCPER

SEQ ID NO: 6 - protein, 22 aa, Artificial Sequence
  synthetic construct; mutant insulin chain
FVNQHLCGSDLVEALYLVCYER

SEQ ID NO: 7 - protein, 22 aa, Artificial Sequence
  synthetic construct; mutant insulin chain
FVNQHLCGSDLVEALYLVCFER

SEQ ID NO: 8 - protein, 22 aa, Artificial Sequence
  synthetic construct; mutant insulin chain
FVNQHLCGSDLVEALYLVCPER

SEQ ID NO: 9 - protein, 22 aa, Artificial Sequence
  synthetic construct; mutant insulin chain
FVNQHLCGSQLVEALYLVCYER

SEQ ID NO: 10 - protein, 22 aa, Artificial Sequence
  synthetic construct; mutant insulin chain
FVNQHLCGSQLVEALYLVCFER

SEQ ID NO: 11 - protein, 22 aa, Artificial Sequence
  synthetic construct; mutant insulin chain
FVNQHLCGSQLVEALYLVCPER

SEQ ID NO: 12 - protein, 21 aa, Artificial Sequence
  synthetic construct; mutant insulin chain
GIVEQCCHRICSLYQLENYCN

SEQ ID NO: 13 - protein, 21 aa, Artificial Sequence
  synthetic construct; mutant insulin chain
GIVEQCCYRICSLYQLENYCN

SEQ ID NO: 14 - protein, 21 aa, Artificial Sequence
  synthetic construct; mutant insulin chain
GIVEQCCKRICSLYQLENYCN

SEQ ID NO: 15 - protein, 20 aa, Artificial Sequence
  synthetic construct; mutant insulin chain
  MISC_FEATURE (4)..(4): gamma-carboxyglutamate
GVVEHCCHRPCSNAEFKKYC

SEQ ID NO: 16 - protein, 23 aa, Artificial Sequence
  synthetic construct; mutant insulin chain
  MISC_FEATURE (5)..(5): hydroxyproline
  MISC_FEATURE (12)..(12): gamma-carboxyglutamate
TFDTPKHRCGSEITNSYMDLCYR

SEQ ID NO: 17 - protein, 22 aa, Artificial Sequence
  synthetic construct; mutant insulin chain
FNVQHLCGSHLVEALYLVCGER

SEQ ID NO: 18 - protein, 22 aa, Artificial Sequence
  synthetic construct; mutant insulin chain
FVNQHLCGSHLVEAYYLVCGER

SEQ ID NO: 19 - protein, 22 aa, Artificial Sequence
  synthetic construct; mutant insulin chain
FVNQHLCGSHLVEALYLVCYER

SEQ ID NO: 20 - protein, 22 aa, Artificial Sequence
  synthetic construct; mutant insulin chain
FVNQHLCGSHLVEAYYLVCYER

SEQ ID NO: 21 - protein, 22 aa, Artificial Sequence
  synthetic construct; mutant insulin chain
  misc_feature (20)..(20): Xaa can be any naturally occurring amino acid
FVNQHLCGSELVEALYLVCXER

SEQ ID NO: 22 - protein, 21 aa, Artificial Sequence
  synthetic construct; mutant insulin chain
GIVEQCCHSICSLYQLENYCN

SEQ ID NO: 23 - protein, 22 aa, Artificial Sequence
  synthetic construct; mutant insulin chain
FVNQHLCGSELVEALYLVCGER

SEQ ID NO: 24 - protein, 21 aa, Artificial Sequence
  synthetic construct; mutant insulin chain
GIVEQCCTRICSLYQLENYCN

SEQ ID NO: 25 - protein, 20 aa, Artificial Sequence
  synthetic construct; mutant insulin chain
  MISC_FEATURE (4)..(4): gamma-carboxyglutamate
GVVEHCCYRPCSNAEFKKFC

SEQ ID NO: 26 - protein, 20 aa, Artificial Sequence
  synthetic construct; mutant insulin chain
  MISC_FEATURE (4)..(4): gamma-carboxyglutamate
GVVEHCCYRPCSNAEFKKFC

SEQ ID NO: 27 - protein, 4 aa, Artificial Sequence
  synthetic construct; peptide
  MISC_FEATURE (1)..(4): Xaa can be any amino acid
XXXX

SEQ ID NO: 28 - protein, 22 aa, Artificial Sequence
  synthetic construct; mutant insulin chain
  MISC_FEATURE (12)..(12): gamma-carboxyglutamate
NSDTPKYRCGSEIPNSYIDLCF

SEQ ID NO: 29 - protein, 22 aa, Artificial Sequence
  synthetic construct; mutant insulin chain
NSDTPKYRCGSDIPNSYMDLCF

SEQ ID NO: 30 - protein, 23 aa, Artificial Sequence
  synthetic construct; mutant insulin chain
  MISC_FEATURE (19)..(19): gamma-carboxyglutamate
NSDTPWNRCGSQITDSYRELCPH

SEQ ID NO: 31 - protein, 24 aa, Artificial Sequence
  synthetic construct; mutant insulin chain
GIVEQCCTSICSLYQLENYCHALQ

SEQ ID NO: 32 - protein, 24 aa, Artificial Sequence
  synthetic construct; mutant insulin chain
GIVEQCCHRICSLYQLENYCHALQ

SEQ ID NO: 33 - protein, 24 aa, Artificial Sequence
  synthetic construct; mutant insulin chain
GIVEQCCYRICSLYQLENYCHALQ

SEQ ID NO: 34 - protein, 24 aa, Artificial Sequence
  synthetic construct; mutant insulin chain
GIVEQCCKRICSLYQLENYCHALQ

SEQ ID NO: 35 - protein, 22 aa, Artificial Sequence
  synthetic construct; mutant insulin chain
FVNQHLCGSELVEALYLVCLER

(19) United States
(12) Patent Application Publication
(10) Pub. No.: US 2022/0389073 A1
(43) Pub. Date: Dec. 8, 2022

(54) Novel Mini-Insulin With Extended C-Terminal A Chain

(71) Applicant: University of Utah Research Foundation, Salt Lake City, UT (US)

(72) Inventors: Danny Hung-Chieh Chou, Salt Lake City, UT (US); Helena Safavi-Hemami, Salt Lake City, UT (US)

(21) Appl. No.: 17/771,064

(22) PCT Filed: Oct. 23, 2020
(86) PCT No.: PCT/US20/57078

§ 371 Date: Apr. 22, 2022

Related U.S. Application Data
(60) Provisional application No. 62/925,617, filed on Oct. 24, 2019.

(51) Int. Cl. C07K 14/62; A61P 5/50

(52) U.S. Cl. CPC C07K 14/62; A61P 5/50

(57) ABSTRACT

Disclosed are peptides comprising an insulin A chain peptide and an insulin B chain peptide, wherein the B chain peptide comprises a substitution at amino acid 10 and amino acid 20. Disclosed are methods of increasing insulin receptor activation in a subject comprising administering a therapeutically effective amount of a disclosed peptide. Disclosed are methods of lowering the blood sugar in a subject comprising administering a therapeutically effective amount of a disclosed peptide. Disclosed are methods of treating type 1 diabetes in a subject comprising administering a therapeutically effective amount of a disclosed. This abstract is intended as a scanning tool for purposes of searching in the particular art and is not intended to be limiting of the present invention.

### CROSS-REFERENCE TO RELATED APPLICATIONS

This application claims the benefit of U.S. Provisional Patent Application No. 62/925,617, filed on Oct. 24, 2019, which is incorporated by reference herein in its entirety.

### BACKGROUND

Maintaining optimal blood glucose levels is effective in delaying or even preventing the long-term complications of diabetes. A major step forward in the care for people with diabetes occurred two decades ago with the introduction of fast-acting insulins. With a more rapid onset and shorter duration of action than injected regular insulin, these synthetic insulin analogues helped people with diabetes achieve tighter blood glucose control while decreasing the incidence of hypoglycemia. Unfortunately, even these fast-acting insulin analogues have limitations. There is still a substantial delay between insulin administration and its onset of action (due to slow diffusion from subcutaneous depots into the bloodstream) (FIG. 1). Additionally, the relatively long duration of action (>4 hours) of these insulin analogues often results in hypoglycemia. Even with the use of fast-acting insulin analogues, glycemic variability continues to be problematic. People with Type 1 diabetes (T1D) only achieve the optimal glucose range (90-130 mg/dl) ˜28% of the time (vs. ˜55% above and 17% below this range).

The availability of an ultrafast-acting insulin (UFI) would enhance compliance with mealtime insulin administration because the UFI could be injected with a meal (or within 5 minutes before), rather than 15-30 minutes before a meal. With a shorter duration of action, a UFI would reduce the risk of postmeal hypoglycemia. Additionally, a UFI would be superior for the rapid correction of hyperglycemia, which may prevent patients from “stacking” insulin injections. Another potential use is with currently available insulin pumps (and artificial pancreas (AP) devices that link glucose sensors to insulin pumps). UFI would increase the performance of insulin pumps and artificial pancreas programs. Although recent clinical studies found that the AP system provided glycemic control that is superior to the current standard of care, with 63% of the time spent in the range of 72-144 mg/dL versus 29% for the conventional pump group, it is still far from what a healthy pancreas can provide (99.2% time <140 mg/dL). Thus, a UFI has the potential to dramatically increase the performance of an AP. A particular benefit would be for the treatment of children/adolescents with type I diabetes (T1D). Insulin therapy in this group represents a difficult challenge due to increasing weight, height, and caloric needs, which lead to hard-to-predict insulin needs, and the mean HbA1 C level for youth with T1D (8.3%) is significantly higher than other groups (7.2%). By providing a tighter action of duration, UFI may be the ideal therapeutic option to address the varying insulin need for youth with T1D. Thus, the development of a UFI with fast onset and short duration of action is an urgent priority.

The clinical need has driven major efforts toward the development of a UFI, although the considerable challenges of this objective are indicated by the failure to develop any approved UFI in the past two decades. The majority of recent efforts have focused on either formulation or mechanical approaches. These include inhaled insulin, intradermal delivery, hyaluronidase-assisted delivery and excipient-based formulations. All these applications use existing insulin analogues and aim to improve its PK/PD to achieve faster acting properties. Although this use of commercially available insulin analogues allows for an accelerated development time frame, it also means that these approaches are likely, at best, to provide only incremental advances.

In contrast, relatively little effort has been applied to developing better insulin analogues. Currently available fast-acting insulin analogues have a relatively slow onset of action because they form dimers, and the rate-limiting step for absorption into the bloodstream is dissociation into monomeric insulin (FIG. 1). Efforts have been stymied by the fundamental problem that insulin dimerization is mediated by aromatic residues at positions 24-26 of the B chain that also bind the insulin receptor, which means that mutations to block dimerization also result in extremely low insulin bioactivity. Development of a UFI will therefore likely involve a radically new approach.

### BRIEF SUMMARY

Disclosed are peptides comprising an insulin A chain peptide and an insulin B chain peptide, wherein the B chain peptide comprises a substitution at amino acid 10 and amino acid 20. In some instances, the substitution at amino acid 20 is G20Y, G20F, or G20P. In some instances, the substitution at amino acid 10 is H10E, H10D or H10Q.

Disclosed are peptides comprising an insulin A chain peptide and an insulin B chain peptide, wherein the B chain peptide comprises a substitution at amino acid 10 and amino acid 20, further comprising at least one substitution in the A chain peptide. In some instances, the at least one substitution in the A chain peptide is T8H, T8Y, T8K, or S9R.

Disclosed are peptides comprising an insulin A chain peptide and an insulin B chain peptide, wherein the B chain peptide comprises a substitution at amino acid 10 and amino acid 20, further comprising at least two substitutions in the A chain peptide. In some instances, the at least two substitutions in the A chain peptide are two of the substitutions selected from: T8H, T8Y, T8K, and S9R.

Disclosed are pharmaceutical compositions comprising a peptide comprising an insulin A chain peptide and an insulin B chain peptide, wherein the B chain peptide comprises a substitution at amino acid 10 and amino acid 20 and a pharmaceutically acceptable carrier.

Disclosed are methods of increasing insulin receptor activation in a subject comprising administering a therapeutically effective amount of a peptide comprising an insulin A chain peptide and an insulin B chain peptide, wherein the B chain peptide comprises a substitution at amino acid 10 and amino acid 20 to a subject in need thereof.

Disclosed are methods of lowering the blood sugar in a subject comprising administering a therapeutically effective amount of a peptide comprising an insulin A chain peptide and an insulin B chain peptide, wherein the B chain peptide comprises a substitution at amino acid 10 and amino acid 20 to a subject in need thereof.

Disclosed are methods of treating type 1 diabetes in a subject comprising administering a therapeutically effective amount of a peptide comprising an insulin A chain peptide and an insulin B chain peptide, wherein the B chain peptide comprises a substitution at amino acid 10 and amino acid 20 to a subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows that insulin monomerization slows absorption rate.

FIG. 2 shows the sequence comparison of Con-Ins-G1 A chain (SEQ ID NO: 15) and human insulin A chain (SEQ ID NO: 1) (top) and Con-Ins-G1 B chain (SEQ ID NO: 16) and human insulin B chain (SEQ ID NO: 2) (bottom). Cysteines are in light blue with the disulfide linkages. The aromatic triplet B24-26 (purple). The B chain of Con-Ins-G1 starts with position “−1” to align with the original human insulin numbering. γ: gamma-carboxyglutamic acid. O:2-hydroxylproline. *: C-terminal amide.

FIG. 3 shows the chemical total synthesis of human DOI insulin. Thr-Ser isopeptide (boxed in red) was used to increase the solubility of insulin A chain. (Cmpd. 1 contains SEQ ID NO: 1; Cmpd. 2 contains SEQ ID NO: 1; Cmpd. 3 contains SEQ ID NO: 17; Cmpd. 4 contains SEQ ID NO: 17; Dex-octapeptide (B23-30) insulin contains both SEQ ID NO: 1 (top) and SEQ ID NO: 17 (bottom)).

FIG. 4 shows the effects of B15 and B20 Tyr on hIR activation. The sequence for each peptide used is also shown. Specifically, the sequences for each peptide used are as follows: human DOI contains SEQ ID NO:1 and SEQ ID NO: 17; B 15Y contains SEQ ID NO: 1 and SEQ ID NO: 18; B 20Y contains SEQ ID NO: 1 and SEQ ID NO: 19; and B 15Y, 20Y contains SEQ ID NO: 1 and SEQ ID NO: 20.

FIG. 5 shows the effects of B10 Glu, B20 Tyr on hIR activation. The sequence for each peptide used is also shown. Specifically, the sequences for each peptide used are as follows: Human Insulin contains SEQ ID NO: 1 and SEQ ID NO: 2; DOI contains SEQ ID NO: 1 and SEQ ID NO: 17; Con-Ins-G1 contains SEQ ID NO: 15 and SEQ ID NO: 16; B 20Y contains SEQ ID NO: 1 and SEQ ID NO: 19; and B 10E, 20Y contains SEQ ID NO: 1 and SEQ ID NO: 3.

FIGS. 6A and 6B show peptide sequences/modified amino acids and effects of B20 residues in activating insulin signaling, respectively. Specifically, FIG. 6A shows SEQ ID NO:1 (top) and SEQ ID NO: 21 (bottom).

FIG. 7 shows the effects of A8 His, A9 Arg on hIR activation. The sequence for each peptide used is also shown. Specifically, the sequences for each peptide used are as follows: Human Insulin contains SEQ ID NO: 1 and SEQ ID NO: 2; Con-Ins-G1 contains SEQ ID NO: 15 and SEQ ID NO: 16; B 10E, 20Y contains SEQ ID NO: 1 and SEQ ID NO: 3: and A 8H, 9R, B 10E, 20Y contains SEQ ID NO: 12 and SEQ ID NO: 3.

FIG. 8 shows the individual effect of A8, A9, B10 and B20 on hIR activation. Specifically, the sequences for each peptide used are as follows: 1: HS+B10E,20Y contains SEQ ID NO: 22 and SEQ ID NO: 3; 2: HR+B10E,L,G contains SEQ ID NO: 12 and SEQ ID NO: 23; 3: TR+B10E,20Y contains SEQ ID NO: 24 and SEQ ID NO: 3; and 4: HR+B20Y contains SEQ ID NO: 12 and SEQ ID NO: 19.

FIG. 9 shows the insulin signaling activation of several venom insulins with similar potencies to Con-Ins G1 (top panel). Sequence alignment of these venom insulins is also shown. Residues at position 9 and 10 in the A chain and 10 and 20 in the B chain are highlighted. γ and * denote post-translational modifications (gamma-carboxyglutmate and C-terminal amidation, respectively). A chain (top to bottom): SEQ ID NO: 1, SEQ ID NO: 15, SEQ ID NO: 25, SEQ ID NO: 26, and SEQ ID NO: 26. B chain (top to bottom): SEQ ID NO: 2, SEQ ID NO: 16, SEQ ID NO: 28, SEQ ID NO: 29, and SEQ ID NO: 30.

FIG. 10 shows an example synthesis strategy for insulin analogs (e.g. having an extended A chain).

FIG. 11 shows example insulin analogs.

FIG. 12 shows mass spectrometry data for insulin analogs.

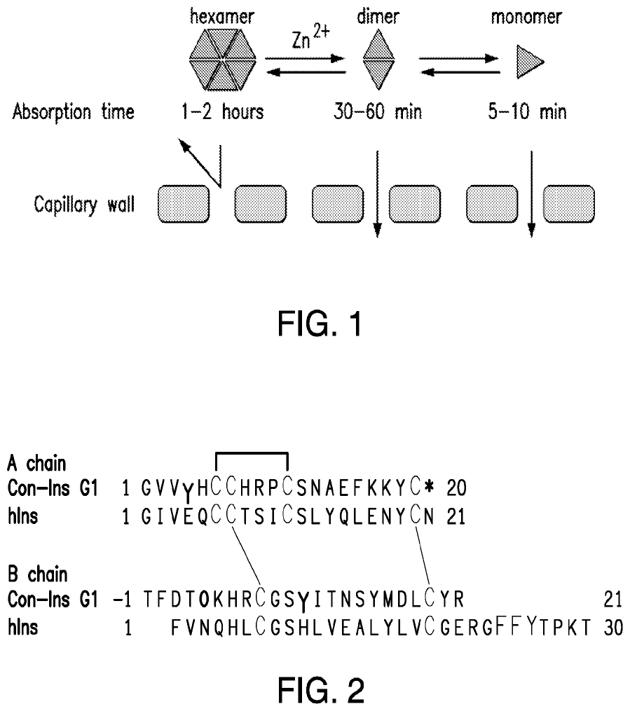

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The terms “A chain peptide” and “B chain peptide” are interchangeable with “insulin A chain peptide” and “insulin B chain peptide.”

The term “therapeutic” refers to a treatment, therapy, or drug that can treat a disease or condition or that can ameliorate one or more symptoms associated with a disease or condition. As used herein, a therapeutic can refer to a therapeutic compound, including, but not limited to proteins, peptides, nucleic acids (e.g. CpG oligonucleotides), small molecules, vaccines, allergenic extracts, antibodies, gene therapies, other biologics or small molecules.

As used herein, the term “subject” or “patient” refers to any organism to which a peptide or composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be type 1 diabetes or any other insulin-related condition.

By a “therapeutically effective amount” of a peptide or pharmaceutical composition as provided herein is meant a sufficient amount of the compound to provide the desired therapeutic effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “therapeutic effective amount.” However, an appropriate “therapeutic effective amount” may be determined by one of ordinary skill in the art using only routine experimentation.

The term amino acid “modification” or “modified” amino acid refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids can be purchased from commercial suppliers, synthesized de novo, or chemically modified or derivatized from naturally occurring amino acids.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids.

A “variant” or “variant thereof” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L- cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. Peptides

Wild type insulin comprises an A chain peptide and a B chain peptide. Wild type human insulin A chain is represented by the sequence GIVEQCCTSICSLYQLENYCN (SEQ ID NO:1). Wild type human insulin B chain is represented by the sequence FVNQHLCGSHLVEALYLVCGERGFFYTPKT (SEQ ID NO:2).

Disclosed are peptides and variants thereof comprising an insulin A chain peptide and an insulin B chain peptide, wherein the B chain peptide comprises a substitution at amino acid 10 and amino acid 20. Disclosed are peptides comprising an A chain peptide and a B chain peptide, wherein the B chain peptide comprises a substitution at amino acid 10 and amino acid 20 compared to wild type human insulin. In some instances, any conservative amino acid substitution can be present at positions 10, 20, or both positions. For example, another hydrophilic amino acid, polar amino acid, or aliphatic amino acid could be substituted at one or both positions.

In some instances of the disclosed peptides, the substitution at amino acid 20 of the B chain peptide can be G20L, G20Y, G20F, or G20P. In some instances, the substitution at amino acid 20 is G20L. In some instances, the substitution at amino acid 20 can be G20P and the peptide further comprises a substitution at amino acid 21, wherein the substitution at amino acid 21 can be G21H. In some instances, the amino acid substitution can be any conservative substitution from glycine.

In some instances of the disclosed peptides, the substitution at amino acid 10 of the B chain peptide can be H10E, H10D or H10Q. In some instances, the substitution at amino acid 10 is H10E. In some instances, the amino acid substitution can be any conservative substitution from histidine.

In some instances, the disclosed insulin analogs have an insulin A chain peptide modified from the wild type sequence. In some instances, the N at position 21 of the insulin A chain peptide can be replaced with the sequence HALQ. For example, the insulin analogs disclosed herein can comprise the amino acid sequence GIVEQCCTSICSLYQLENYCHALQ (SEQ ID NO:31).

In some instances, both the insulin A chain peptide and the B chain peptide can contain substitutions compared to wild type insulin. Thus, in some aspects, the insulin A chain peptide and the B chain peptide can be variants of wild type insulin. Disclosed are peptides comprising an insulin A chain peptide and an insulin B chain peptide, wherein the B chain peptide comprises at least a substitution at amino acid 10 and amino acid 20 and the A chain peptide can comprise the sequence of SEQ ID NO:31 . In some instances, the insulin analog can further comprise at least one amino acid substitution to SEQ ID NO:31. In some instances, the at least one substitution can be found at position 8 or 9. In some instances, the at least one substitution in the A chain peptide can be T8H, T8Y, T8K, or S9R. In some instances, any conservative amino acid substitution can be present at position 8 or 9 or both positions. For example, another hydrophilic amino acid could be substituted or other polar amino acids could be substituted.

Disclosed are peptides comprising an insulin A chain peptide and an insulin B chain peptide, wherein the B chain peptide comprises a substitution at amino acid 10 and amino acid 20 and further comprising at least two substitutions in the A chain peptide. In some instances, the at least two substitutions can be found at positions 8 and 9. In some instances, the at least two substitutions in the A chain peptide can be selected from: T8H, T8Y, T8K, and S9R. In some instances, any conservative amino acid substitution can be present at position 8 or 9 or both positions. For example, another hydrophilic amino acid could be substituted or other polar amino acids could be substituted at one or both positions.

In some instances, the B chain peptide is lacking one or more, up to eight, of the C-terminal amino acids compared to wild type. Thus, the disclosed peptides can be des-octapeptide insulin peptides (missing the last 8 amino acids of the C-terminus of the human insulin B chain). For example, in some instances the disclosed peptides can have a B chain peptide that comprises the sequence of FVNQHLCGSELVEALYLVCYER (SEQ ID NO:3), FVNQHLCGSELVEALYLVCFER (SEQ ID NO:4), FVNQHLCGSELVEALYLVCPER (SEQ ID NO:5), FVNQHLCGSDLVEALYLVCYER (SEQ ID NO:6), FVNQHLCGSDLVEALYLVCFER (SEQ ID NO:7), FVNQHLCGSDLVEALYLVCPER (SEQ ID NO:8), FVNQHLCGSQLVEALYLVCYER (SEQ ID NO:9), FVNQHLCGSQLVEALYLVCFER (SEQ ID NO:10), FVNQHLCGSQLVEALYLVCPER (SEQ ID NO:11), or variant thereof.

In some instances, the disclosed peptides can have an A chain comprising the sequence of GIVEQCCHRICSLYQLENYCHALQ (SEQ ID NO:32), GIVEQCCYRICSLYQLENYCHALQ (SEQ ID NO:33), GIVEQCCKRICSLYQLENYCHALQ (SEQ ID NO:34) or variant thereof. In some instances, the disclosed peptides can have an A chain comprising the sequence of GIVEQCCHRICSLYQLENYCN (SEQ ID NO:12), GIVEQCCYRICSLYQLENYCN (SEQ ID NO:13), GIVEQCCKRICSLYQLENYCN (SEQ ID NO:14), or variant thereof.

In some instances of the disclosed peptides, the A chain peptide and B chain peptide can be bonded via at least one disulfide bond. In some instances, the A chain peptide and B chain peptide can be bonded via at least two disulfide bonds.

In some instances, the disclosed peptides are monomers. In other words, in some instances, the disclosed peptides are less likely to form dimers, tetramers, hexamers, etc.

In some instances of the disclosed peptides, the insulin A chain peptide can be at least 70% identical to wild type human insulin A chain peptide. In some instances, the insulin A chain peptide can be at least 60, 65, 70, 75, 80, 85, 90, 95, 99% identical to wild type human insulin A chain peptide. In some instances, the percent identity can be reached by the deletion of one or more amino acids from the N-terminus or C-terminus end of the disclosed peptides. In some instances of the disclosed peptides, the insulin A chain peptide can be at least 70% identical to SEQ ID NO:31. In some instances, the insulin A chain peptide can be at least 60, 65, 70, 75, 80, 85, 90, 95, 99% identical to SEQ ID NO:31. In some instances, the percent identity can be reached by the deletion or substitution of one or more amino acids other than the C-terminal HALQ.

In some instances of the disclosed peptides, the insulin B chain peptide can be at least 70% identical to wild type human insulin B chain peptide. In some instances, the insulin B chain peptide can be at least 60, 65, 70, 75, 80, 85, 90, 95, 99% identical to wild type human insulin B chain peptide. In some instances, the percent identity can be reached by the deletion of one or more amino acids from the N-terminus or C-terminus end of the disclosed peptides.

In some instances, the disclosed peptides can comprise one or more unnatural amino acids, modified amino acids or synthetic amino acid analogues. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, cyclopentylalanine, β-alanine, fluoro-amino acids, designer amino acids such as β-methyl amino acids, Cα-methyl amino acids, Nα-methyl amino acids, and amino acid analogues in general. Also included within the scope are peptides which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the peptide.

In some aspects of the disclosed peptides, provided are therapeutic proteins having an A chain peptide bonded to a B chain peptide via at least one disulfide bond, wherein the A chain comprises the sequence of GIVEQCCHRICSLYQLENYCHALQ (SEQ ID NO:31), and wherein the B chain peptide comprises the sequence of FVNQHLCGSELVEALYLVCLER (SEQ ID NO:35). It is appreciated that the disclosed therapeutic proteins can be employed in pharmaceutical compositions and used in connection with treatment of disorders including diabetes.

In further instances of the disclosed peptides, provided are therapeutic proteins having an A chain peptide bonded to a B chain peptide via at least one disulfide bond, wherein the A chain comprises the sequence of GIVEQCCHRICSLYQLENYCN (SEQ ID NO: 12), and wherein the B chain peptide comprises the sequence of FVNQHLCGSELVEALYLVCYER (SEQ ID NO: 3). It is appreciated that the disclosed therapeutic proteins can be employed in pharmaceutical compositions and used in connection with treatment of disorders including diabetes.

### C. Pharmaceutical Compositions

Disclosed are pharmaceutical compositions comprising one or more of the disclosed peptides or variants thereof and a pharmaceutically acceptable carrier.

In some instances, the disclosed peptides or variants thereof can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In one aspect, disclosed are pharmaceutical compositions comprising any of the disclosed peptides described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, disclosed are pharmaceutical composition comprising a pharmaceutically acceptable carrier or diluent and, as active ingredient, a therapeutically effective amount of a disclosed compound, a product of a disclosed method of making, a pharmaceutically acceptable salt, solvate, or polymorph thereof, a hydrate thereof, a solvate thereof, a polymorph thereof, or a stereochemically isomeric form thereof. In a further aspect, a disclosed compound, a product of a disclosed method of making, a pharmaceutically acceptable salt, solvate, or polymorph thereof, a hydrate thereof, a solvate thereof, a polymorph thereof, or a stereochemically isomeric form thereof, or any subgroup or combination thereof may be formulated into various pharmaceutical forms for administration purposes.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention.

The pharmaceutically acceptable acid and base addition salts as mentioned hereinabove or hereinafter are meant to comprise the therapeutically active non-toxic acid and base addition salt forms which the disclosed compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The disclosed compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Thus, the pharmaceutical compositions of this invention can include a pharmaceutically acceptable carrier and a compound or a pharmaceutically acceptable salt of the compounds of the invention. By “pharmaceutically acceptable” is meant a material or carrier that would be selected to minimize any degradation of the active ingredient and to minimize any adverse side effects in the subject, as would be well known to one of skill in the art. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration is preferred, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a peptide such as sPRR (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions that require increasing insulin receptor activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The composition can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific composition employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

In a further aspect, a dosage can be 100U-300U vial, for example, a 100U-200U vial, a 200U-300U vial, or a 150U-250U vial. It can be taken once a day or multiple times a day. In some instances it can be taken daily, weekly or monthly.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present invention is further directed to a method for the manufacture of a medicament for modulating insulin receptor activity (e.g., treatment of type 1 diabetes) in mammals (e.g., humans) comprising combining one or more disclosed peptides or compositions with a pharmaceutically acceptable carrier or diluent. Thus, in one aspect, the invention relates to a method for manufacturing a medicament comprising combining at least one disclosed peptide with a pharmaceutically acceptable carrier or diluent.

The disclosed pharmaceutical compositions can further comprise other therapeutically active compounds, which are usually applied in the treatment of insulin-related conditions.

It is understood that the disclosed compositions can be prepared from the disclosed peptides. It is also understood that the disclosed compositions can be employed in the disclosed methods of using.

As already mentioned, the invention relates to a pharmaceutical composition comprising a therapeutically effective amount of a disclosed peptide, a pharmaceutically acceptable salt, solvate, or polymorph thereof, a hydrate thereof, a solvate thereof, a polymorph thereof, and a pharmaceutically acceptable carrier. Additionally, the invention relates to a process for preparing a pharmaceutical composition, characterized in that a pharmaceutically acceptable carrier is intimately mixed with a therapeutically effective amount of a disclosed peptide.

As already mentioned, the invention also relates to a pharmaceutical composition comprising a disclosed peptide, a pharmaceutically acceptable salt, solvate, or polymorph thereof, and one or more other drugs in the treatment, prevention, control, amelioration, or reduction of risk of diseases or conditions for a disclosed peptide or the other drugs may have utility as well as to the use of such a composition for the manufacture of a medicament. The present invention also relates to a combination of disclosed peptides, a pharmaceutically acceptable salt, solvate, or polymorph thereof, and an anti-cancer therapeutic agent. In various further aspects, the present invention also relates to a combination of disclosed peptides, a pharmaceutically acceptable salt, solvate, or polymorph thereof. The present invention also relates to such a combination for use as a medicine. The different drugs of such a combination or product may be combined in a single preparation together with pharmaceutically acceptable carriers or diluents, or they may each be present in a separate preparation together with pharmaceutically acceptable carriers or diluents.

In some instances, the disclosed peptides can be administered in an amount of 10-300 μg/kg/day. In some instances, the dosing regimen can include a single administration of one or more of the disclosed peptides. In some instances, the dosing regimen can include administering one or more of the disclosed peptides once a week, twice a week, three times a week, four times a week, five times a week, six times a week, or seven times a week for 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 38, 40, 42, 44, 46, 48, 50 or 52 weeks.

### D. Methods of Increasing Insulin Receptor Activation

Disclosed are methods of increasing insulin receptor activation in a subject comprising administering a therapeutically effective amount of any one of the disclosed peptides or pharmaceutical compositions to a subject in need thereof. In some instances, a subject in need thereof can be a subject known to have decreased insulin receptor activation compared to a standard activation level. In some instances, a standard activation level of insulin receptor activation can be based on established levels in healthy individuals. In some instances, a standard activation level of insulin receptor activation can be based on established levels in the subject being treated prior to the determination of a need for increased insulin receptor activation.

For example, disclosed are methods of increasing insulin receptor activation in a subject comprising administering a therapeutically effective amount of a peptide comprising an insulin A chain peptide and an insulin B chain peptide, wherein the B chain peptide comprises a substitution at amino acid 10 and amino acid 20 and the A chain peptide comprises the sequence of SEQ ID NO:X to a subject in need thereof. In some instances, the substitution at amino acid 20 of the B chain peptide can be G20L, G20Y, G20F, or G20P. In some instances of the disclosed peptides, the substitution at amino acid 10 of the B chain peptide can be H10E, H10D or H10Q. In some instances, any combination of the B chain substitutions at amino acid 10 and 20 can be present. In some instance, the A chain of the administered peptide can also comprise at least one substitution. In some instances, the at least one amino acid substitution is a substitution from the sequence of or compared to SEQ ID NO:31. For example, in some instances, the at least one substitution in the A chain peptide can be T8H, T8Y, T8K, or S9R. In some instances, the amino acid substitution can be present at position 8 or 9 or both positions. Thus, in some instances, any combination of the disclosed B chain peptide substitutions and A chain peptide substitutions can be present.

### E. Methods of Lowering Blood Sugar

Disclosed are methods of lowering the blood sugar in a subject comprising administering a therapeutically effective amount of any one of the disclosed peptides or pharmaceutical compositions to a subject in need thereof.

In some instances, a subject in need thereof can be a subject known to have increased blood sugar compared to a standard blood sugar level. In some instances, a standard activation level of insulin receptor activation can be based on established levels in healthy individuals. In some instances, a standard activation level of insulin receptor activation can be based on established levels in the subject being treated prior to the determination of a need for increased insulin receptor activation.

For example, disclosed are methods of lowering the blood sugar in a subject comprising administering a therapeutically effective amount of a peptide comprising an insulin A chain peptide and an insulin B chain peptide, wherein the B chain peptide comprises a substitution at amino acid 10 and amino acid 20 and the A chain peptide comprises the sequence of SEQ ID NO:31 to a subject in need thereof. In some instances, the substitution at amino acid 20 of the B chain peptide can be G20L, G20Y, G20F, or G20P. In some instances of the disclosed peptides, the substitution at amino acid 10 of the B chain peptide can be H10E, H10D or H10Q. In some instances, any combination of the B chain substitutions at amino acid 10 and 20 can be present. In some instance, the A chain of the administered peptide can also comprise at least one substitution. In some instances, the at least one amino acid substitution is compared to SEQ ID NO:31. For example, in some instances, the at least one substitution in the A chain peptide can be T8H, T8Y, T8K, or S9R. In some instances, the amino acid substitution can be present at position 8 or 9 or both positions. Thus, in some instances, any combination of the disclosed B chain peptide substitutions and A chain peptide substitutions can be present.

### F. Methods of Treating

Disclosed are methods of treating insulin-related conditions in a subject comprising administering a therapeutically effective amount of any one of the disclosed peptides or pharmaceutical compositions to a subject in need thereof. An insulin-related condition can be hyperglycemia, insulin resistance, type-1 diabetes, gestation diabetes or type-2 diabetes. A subject in need thereof can be any subject that would benefit from an insulin-related condition treatment or therapy.

Disclosed are methods of treating type 1 diabetes in a subject comprising administering a therapeutically effective amount of any one of the disclosed peptides or pharmaceutical compositions to a subject in need thereof. A subject in need thereof can be any subject that would benefit from a type 1 diabetes treatment or therapy.

In some instances, the subject has been diagnosed with type 1 diabetes prior to administering the peptide. In some instances, the subject has been diagnosed with being at risk for developing type 1 diabetes prior to administering the peptide.

For example, disclosed are methods of treating type 1 diabetes in a subject comprising administering a therapeutically effective amount of a peptide comprising an insulin A chain peptide and an insulin B chain peptide, wherein the B chain peptide comprises a substitution at amino acid 10 and amino acid 20 and the A chain peptide comprises the sequence of SEQ ID NO:31 to a subject in need thereof. In some instances, the substitution at amino acid 20 of the B chain peptide can be G20L, G20Y, G20F, or G20P. In some instances of the disclosed peptides, the substitution at amino acid 10 of the B chain peptide can be H10E, H10D or H10Q. In some instances, any combination of the B chain substitutions at amino acid 10 and 20 can be present. In some instance, the A chain of the administered peptide can also comprise at least one substitution. In some instances, the at least one amino acid substitution is compared to SEQ ID NO:31. For example, in some instances, the at least one substitution in the A chain peptide can be T8H, T8Y, T8K, or S9R. In some instances, the amino acid substitution can be present at position 8 or 9 or both positions. Thus, in some instances, any combination of the disclosed B chain peptide substitutions and A chain peptide substitutions can be present.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed peptides.

### EXAMPLES

### A. Insulin Analogs

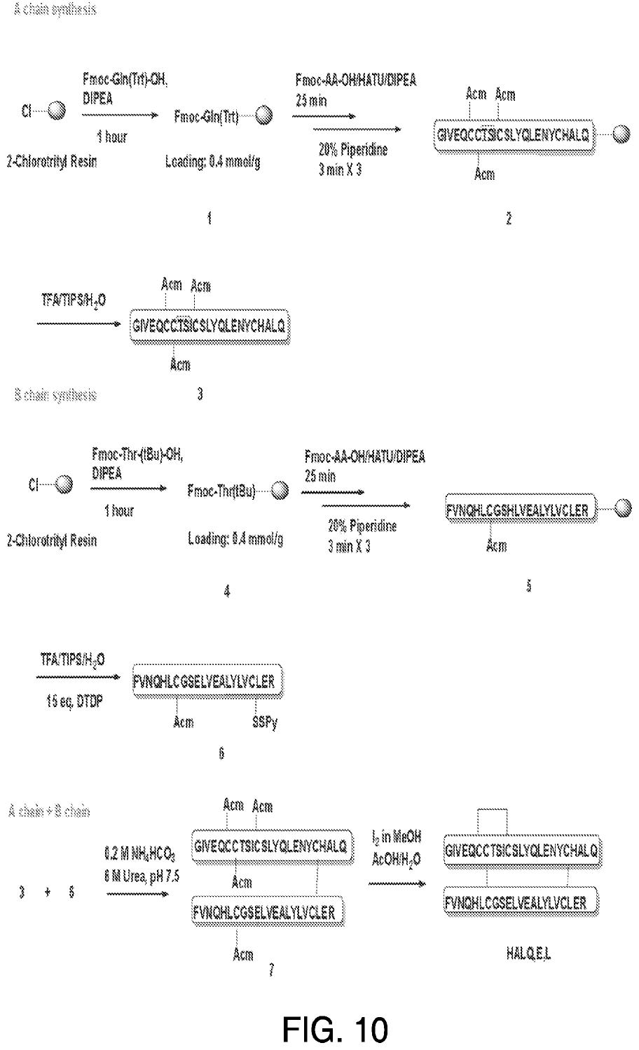

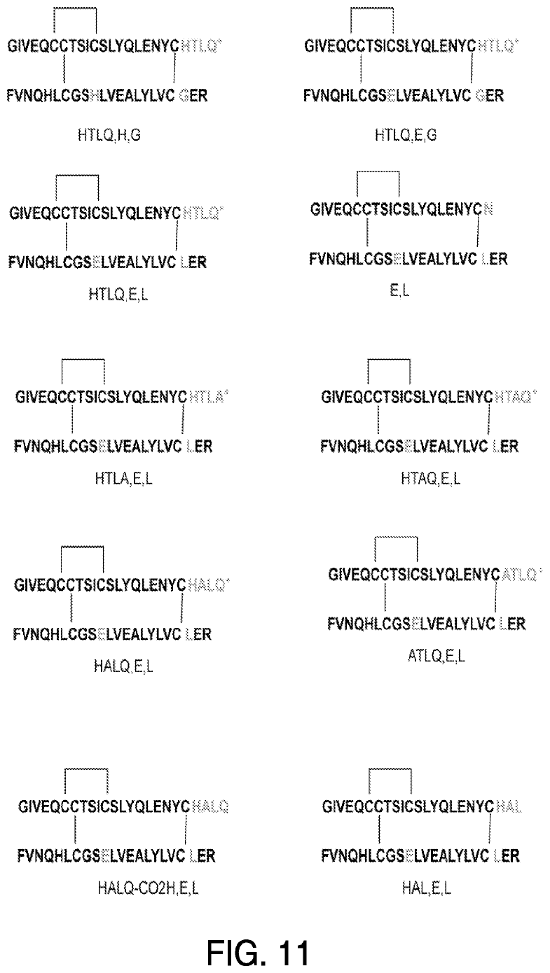

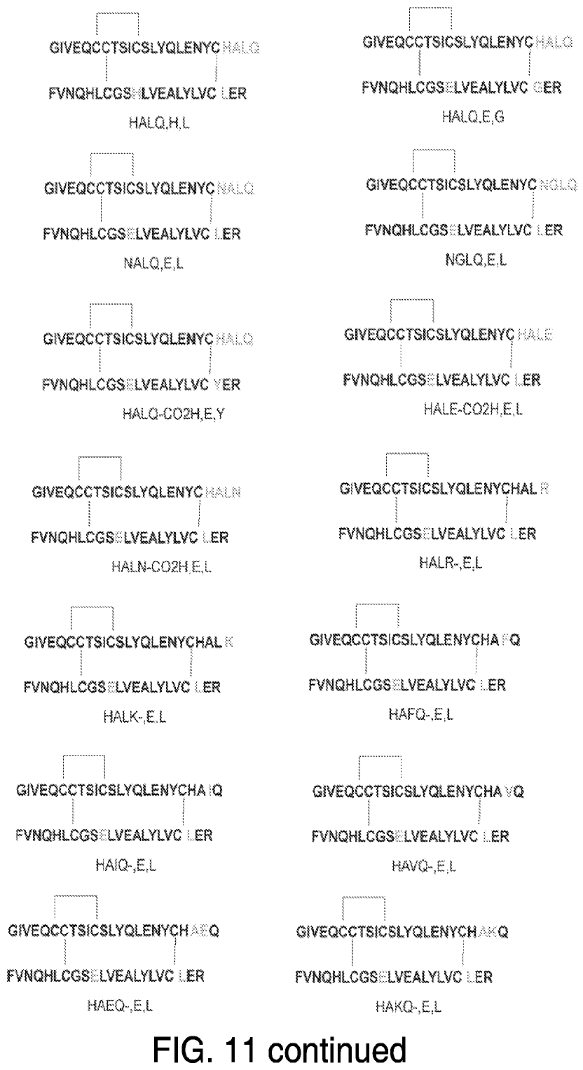

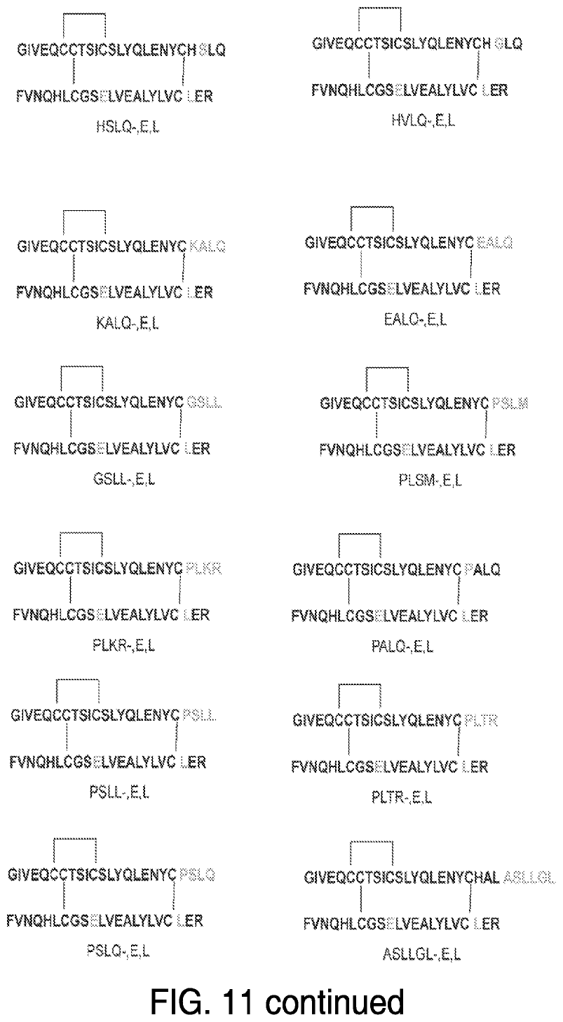

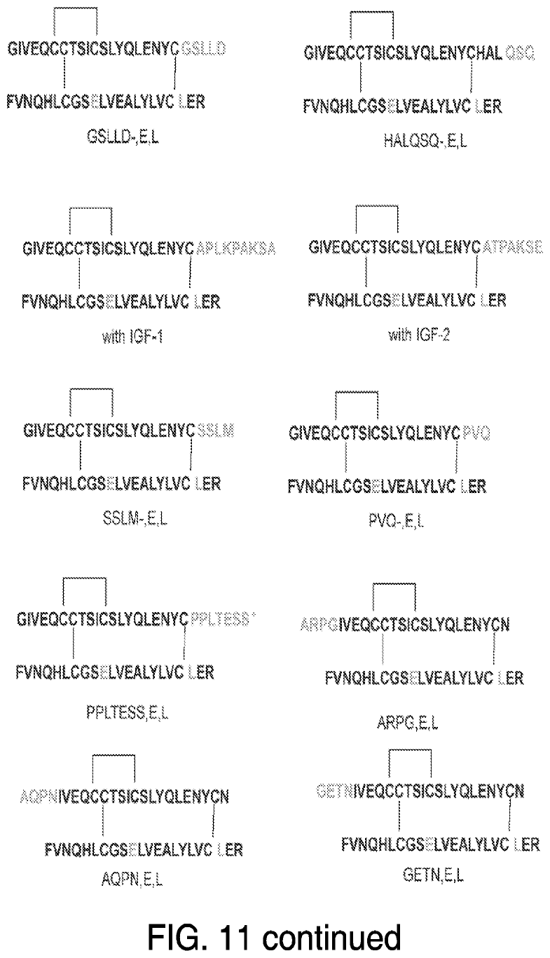

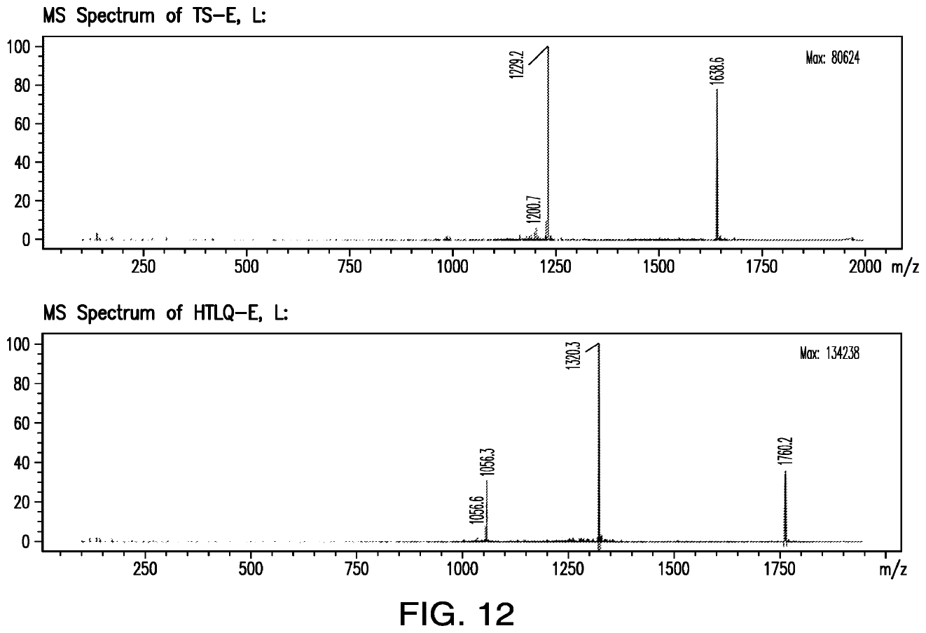

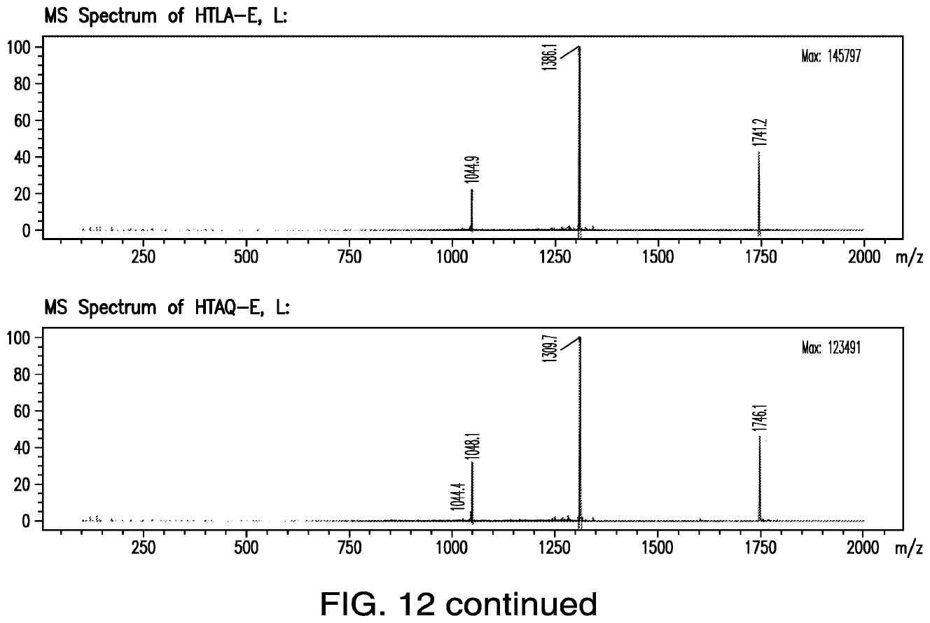

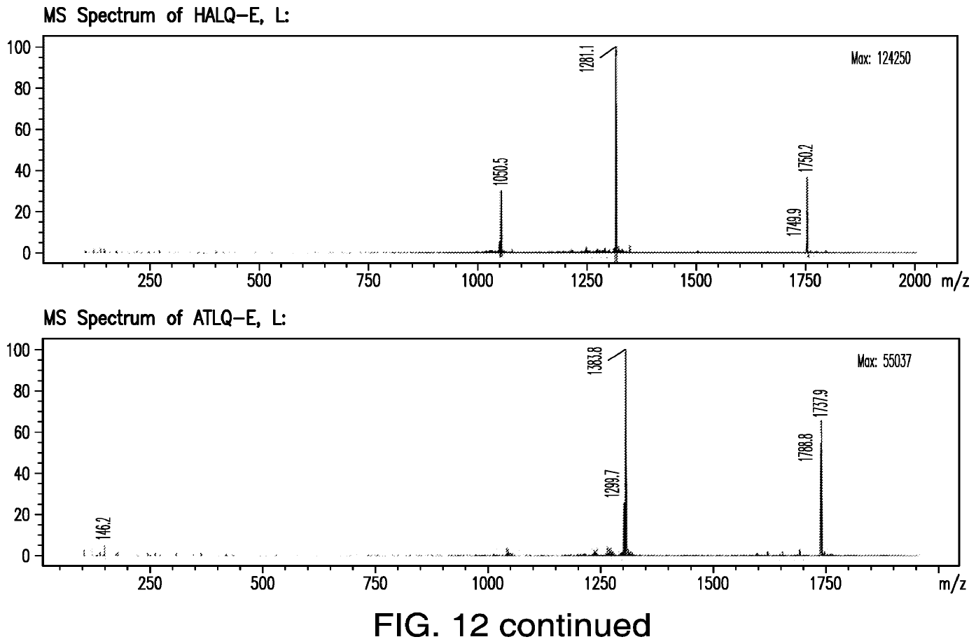

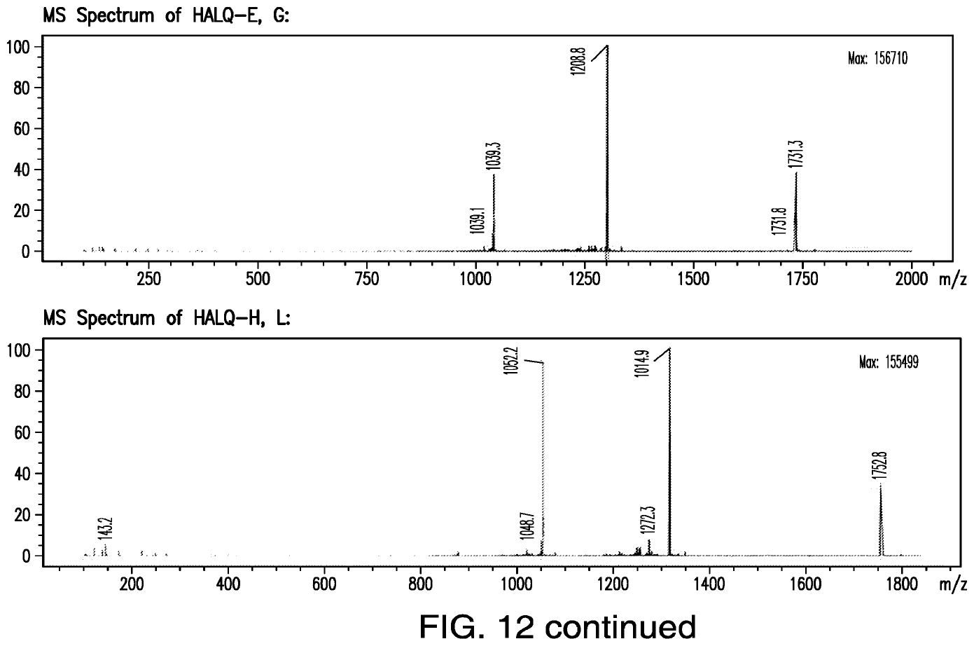

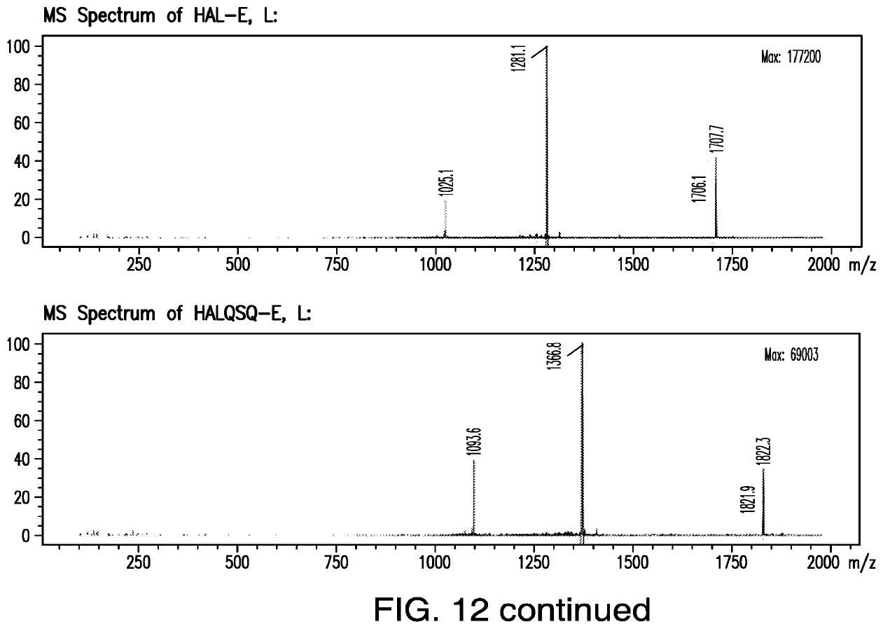

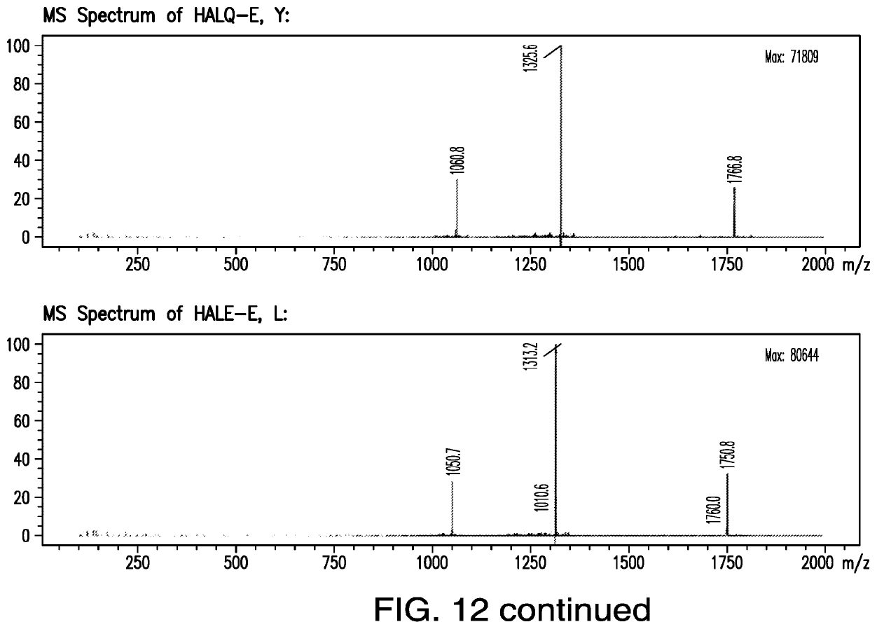

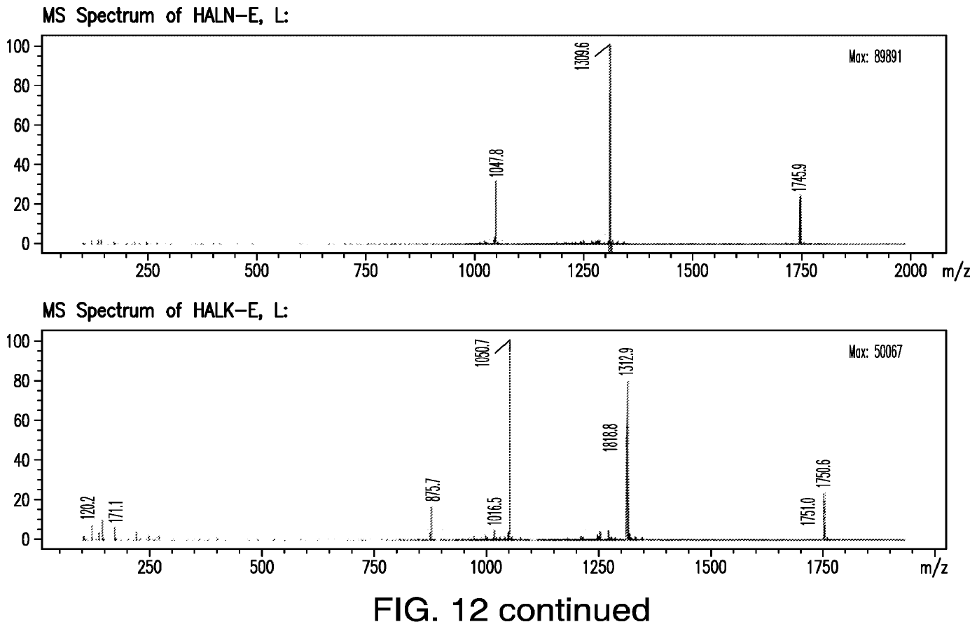

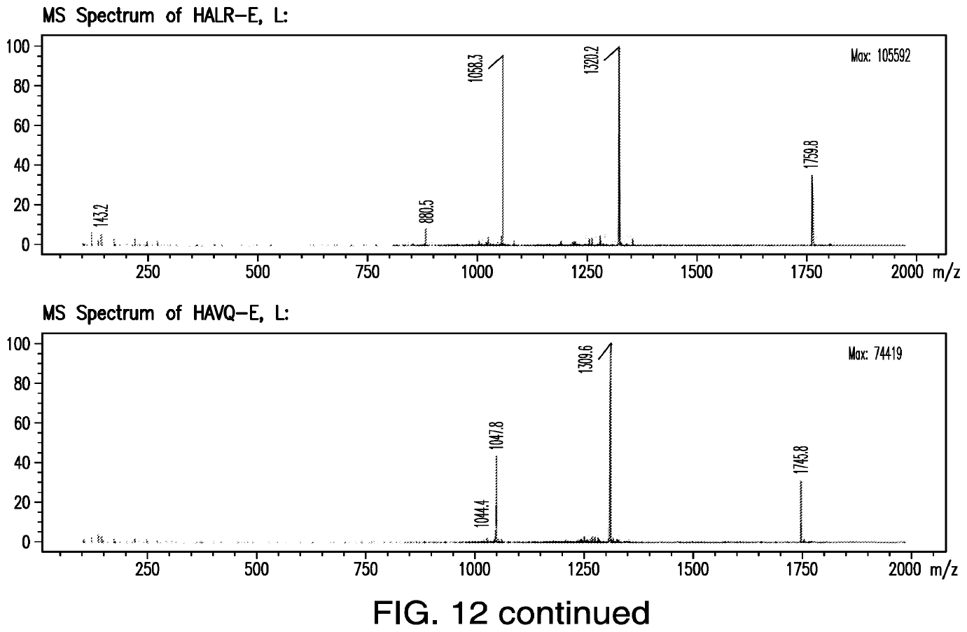

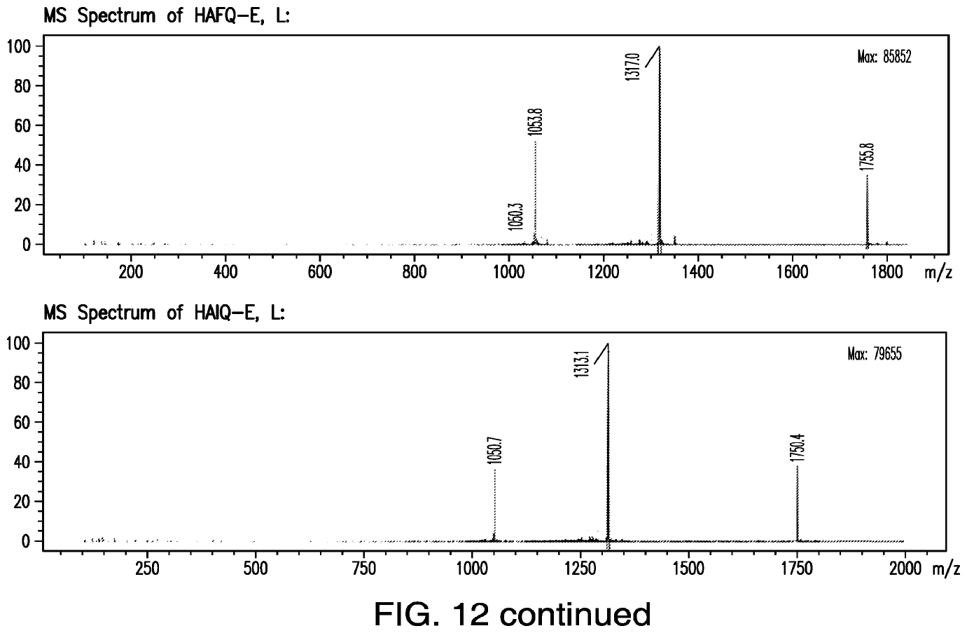

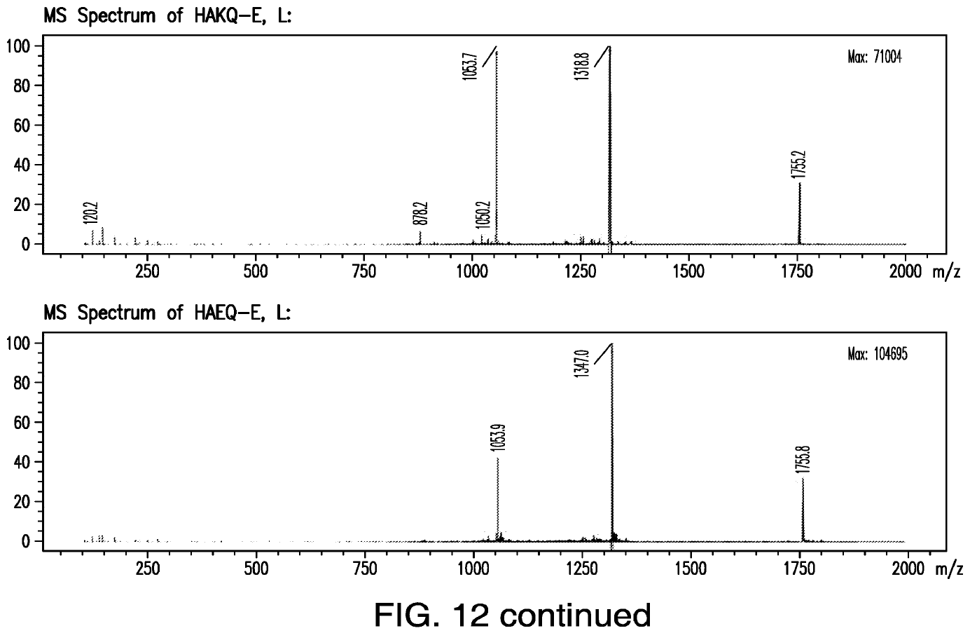

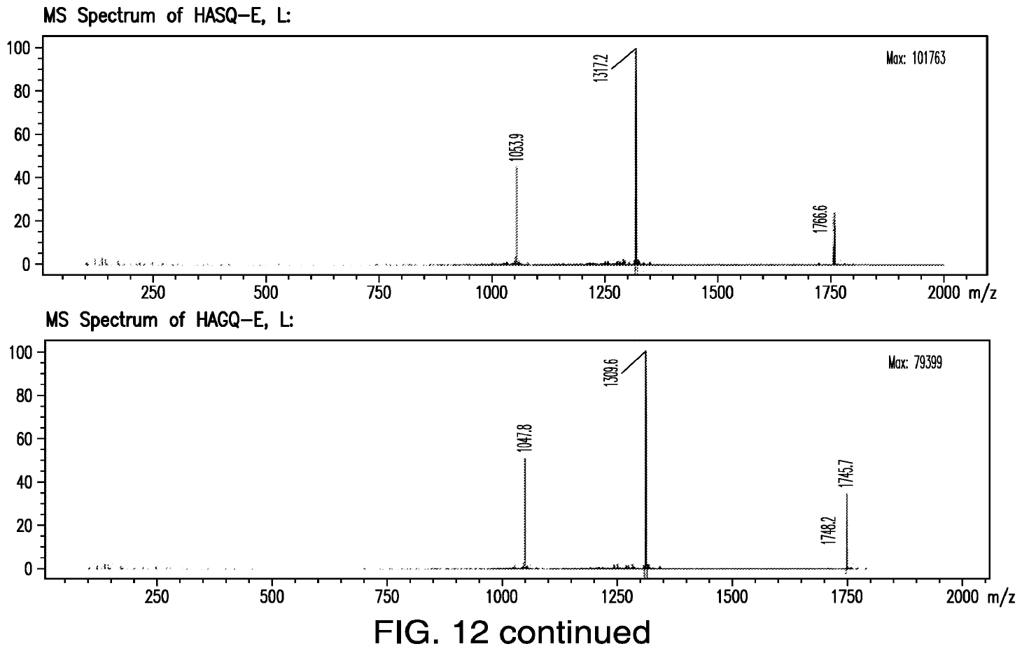

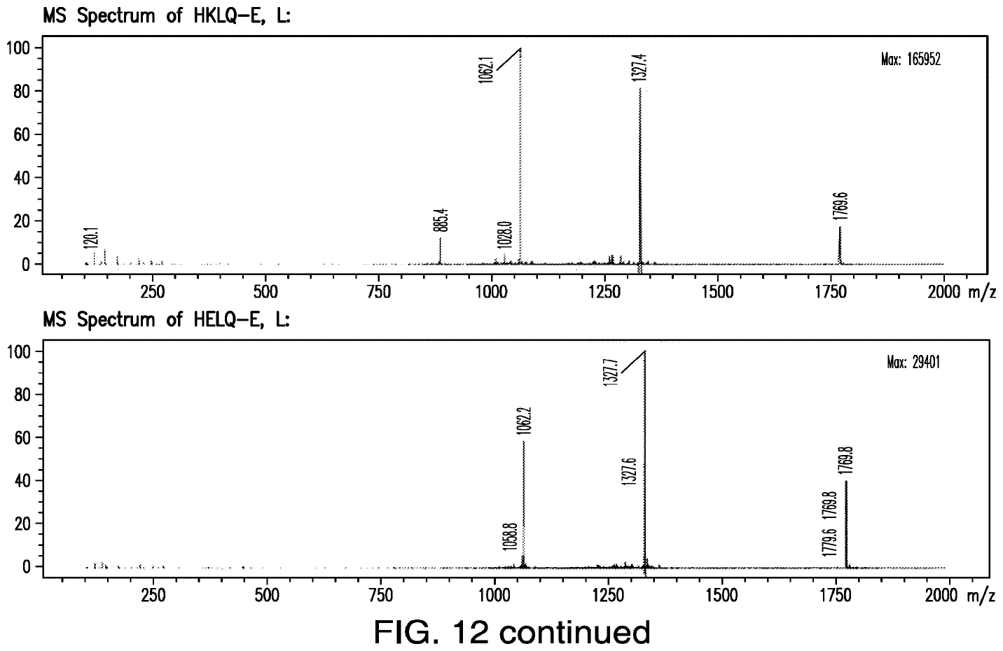

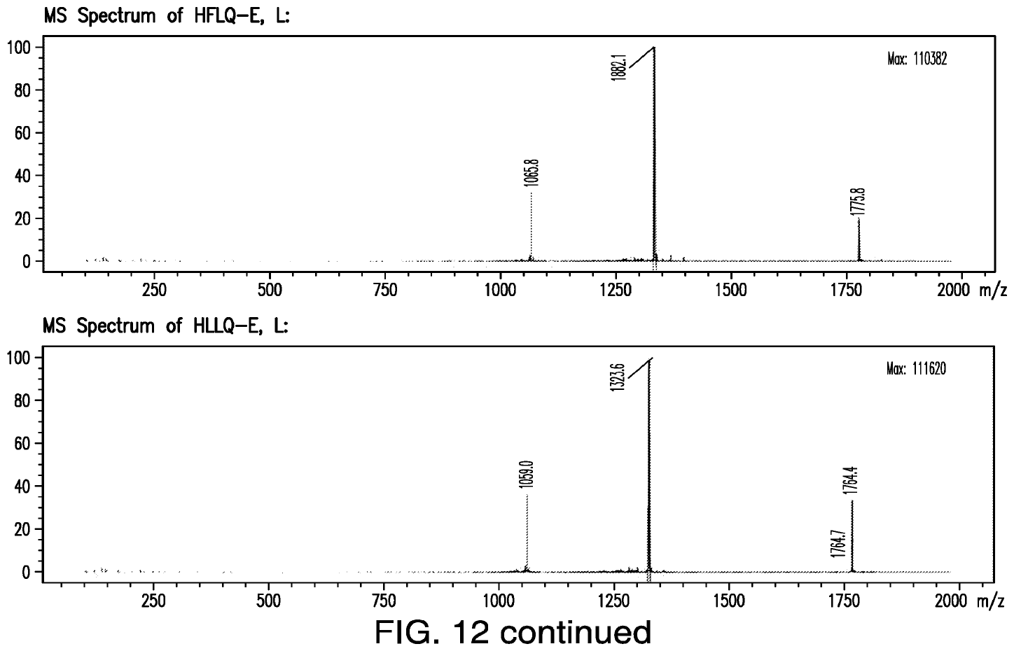

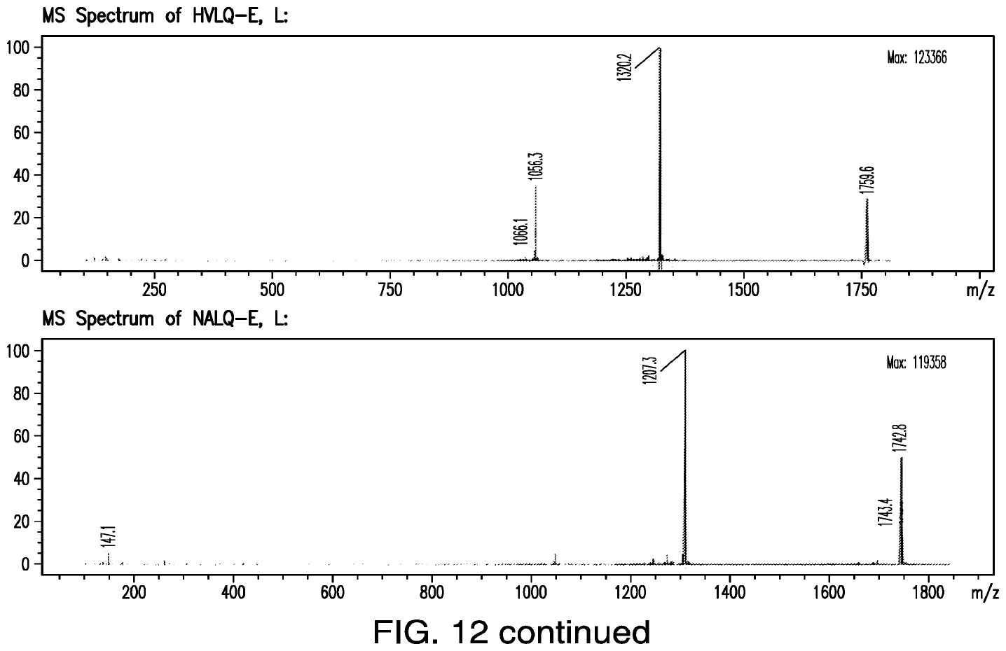

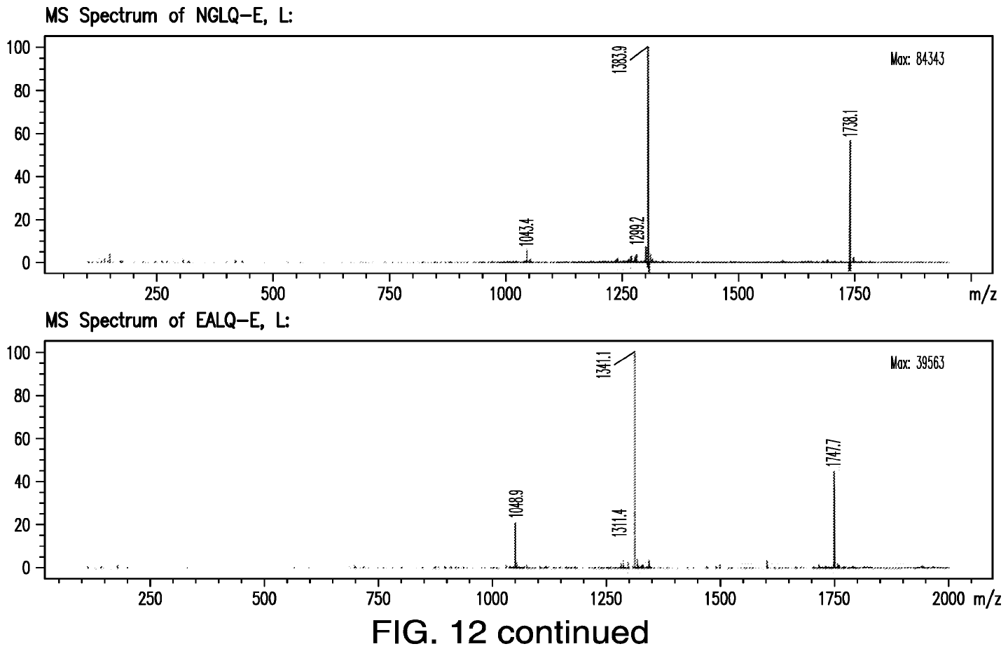

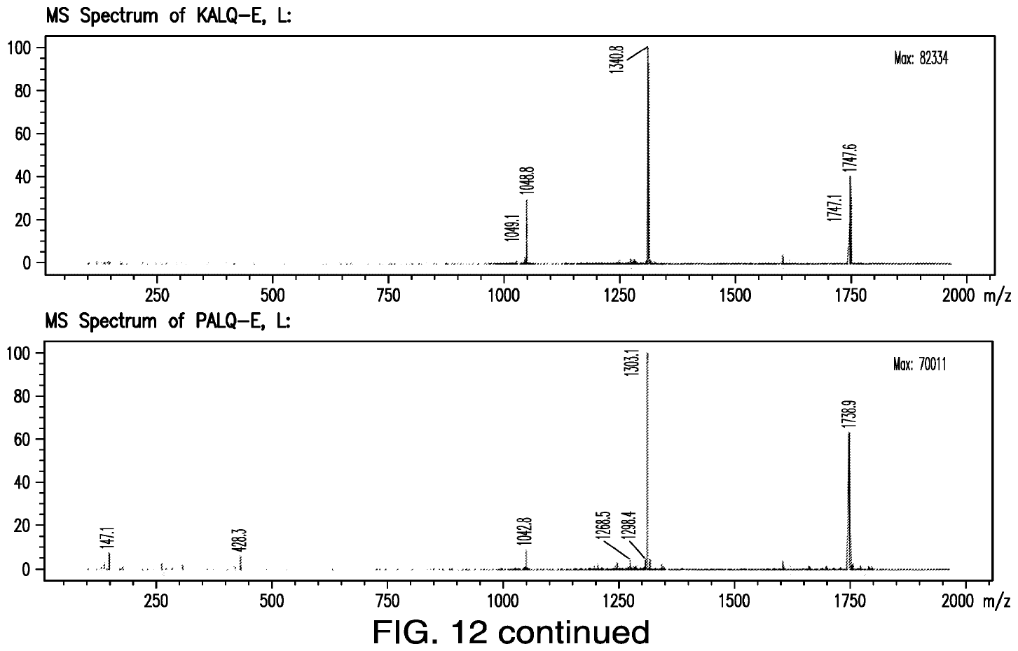

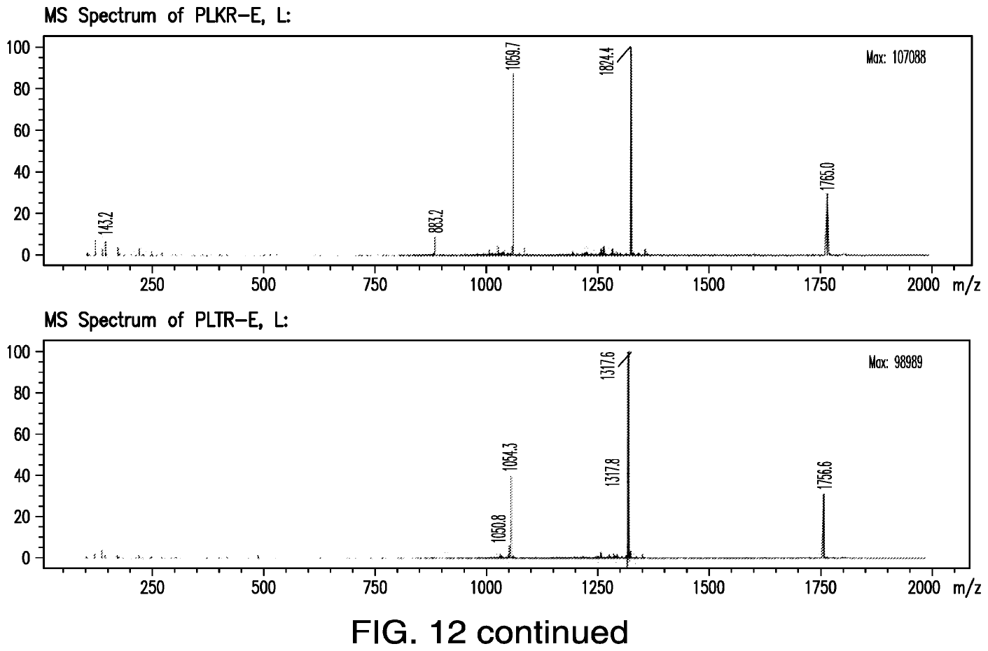

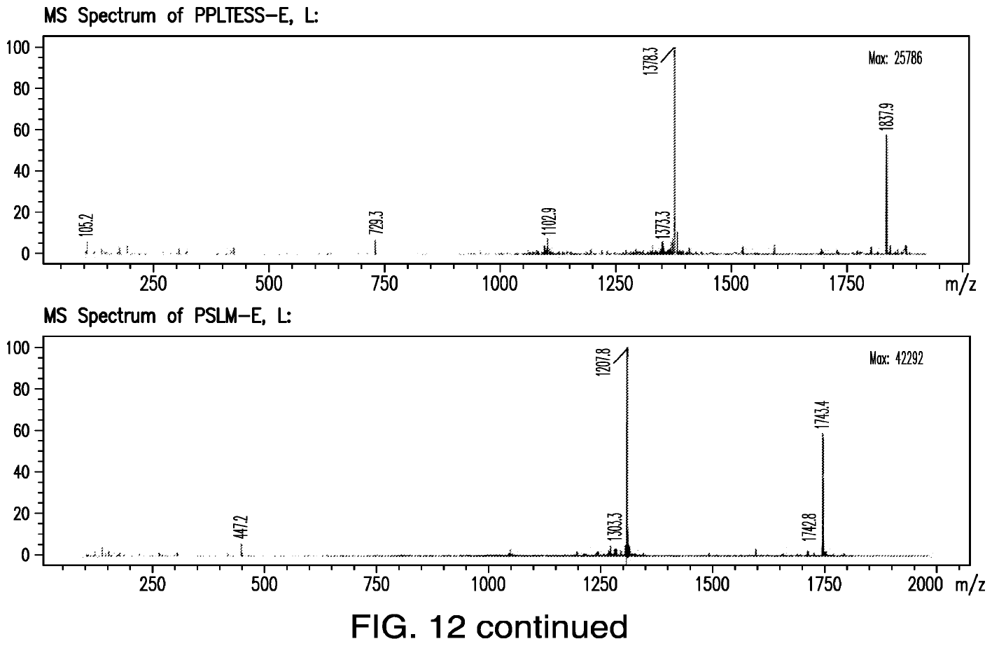

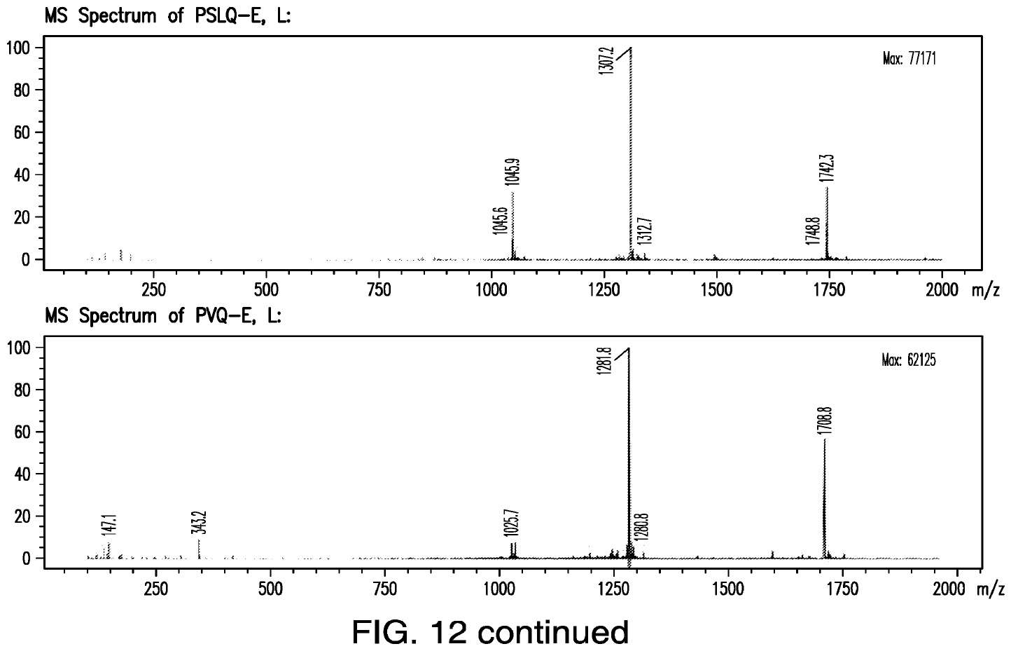

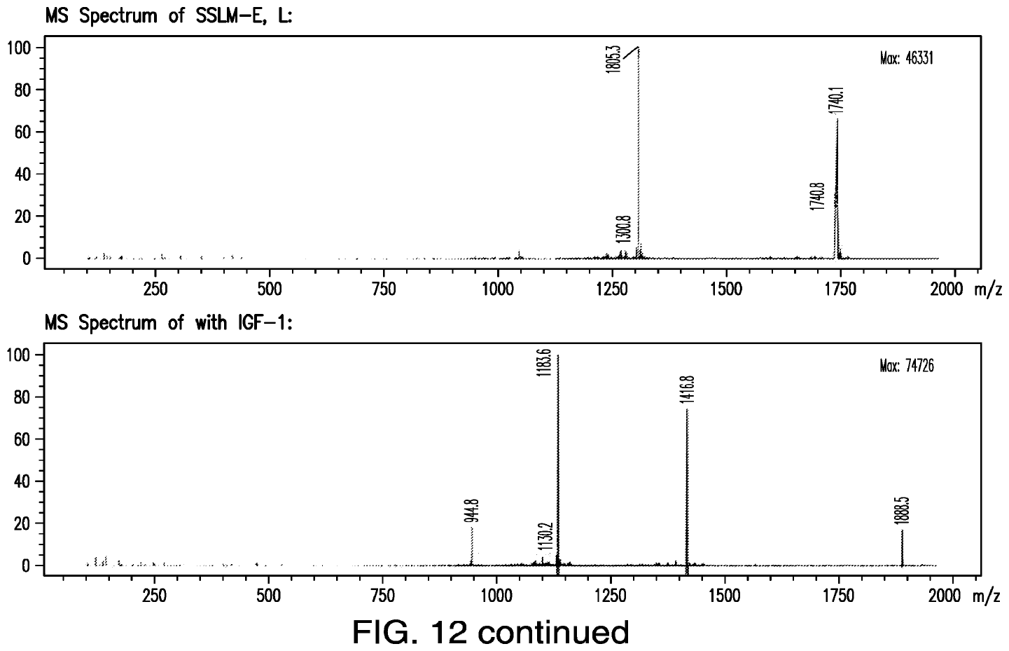

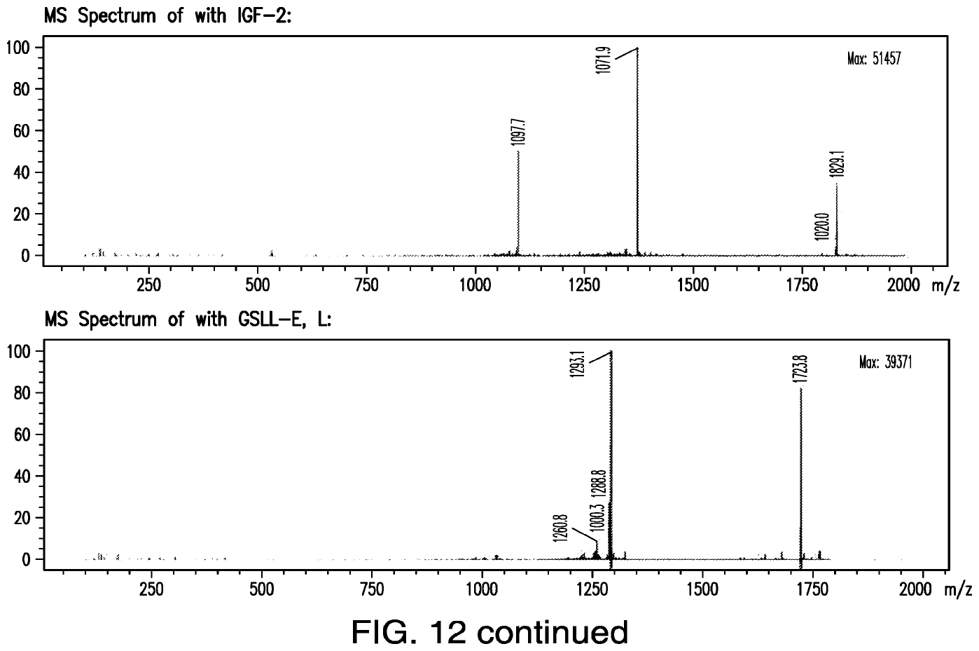

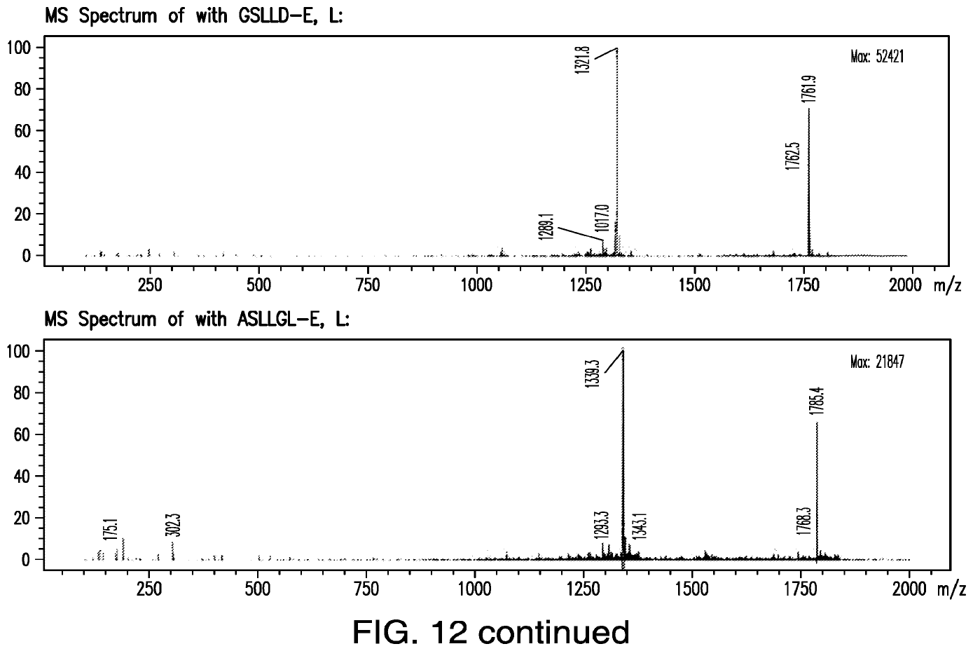

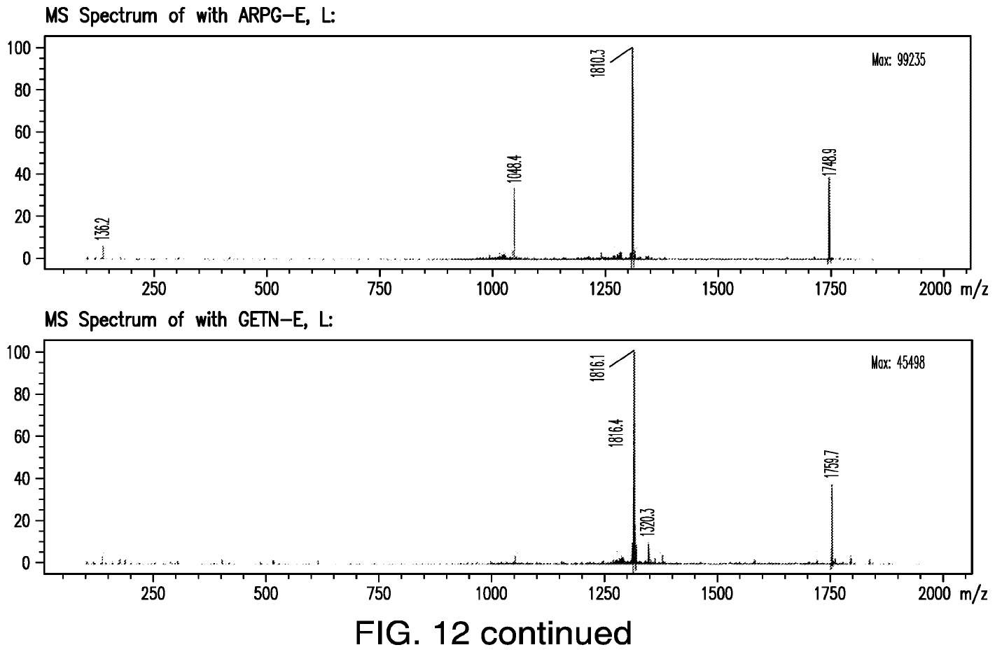

A total of 46 insulin analogs with truncated C-terminal B chain and extended C-terminal A chain were synthesized. Examples of the synthetic route and detailed sequence of each insulin were reported and illustrated in FIGS. 10-12

### B. Research Strategy for New Insulin Analogs

As described herein, the recent discovery of a monomeric insulin variant (Con-Ins-G1) in the venom of a predatory snail has helped propel the research behind the disclosed peptides. Methods have been developed and data has been obtained that explain how Con-Ins-G1 both avoids dimerization and maintains receptor binding and insulin signaling, and thereby acts very quickly. Furthermore, insights from fundamental discoveries have been used to develop a protein that only differs from the sequence of human insulin at four amino acid positions yet is monomeric, fast acting, and displays potency comparable to that of authentic human insulin.

The insights gained from study of Con-Ins-G1 were used to develop a monomeric human insulin that displays only four amino acid substitutions from the human “shortened” protein.

To circumvent the constraints of human insulin's structure, solutions were taken from nature: fish-hunting cone snails, Conus geographus, have evolved the use of specialized insulin from their venom that induces paralyzing hypoglycemic shock in fish within seconds. The sequence of venomous insulin, Con-Ins- G1, was elucidated using a combination of genome sequencing and mass spectrometry (FIG. 2). Notably, four post-translational modifications were observed: A4 Glu and B10 Glu to gammacarboxyglutamic acid, Gla; B3 Pro to 2-hydroxylproline, and a C-terminal amide on the A-chain. Due to the low abundance of this venomous insulin, it cannot be isolated from the animal. Instead, a synthetic analogue (sCon-Ins-G1) was obtained in which, for ease of synthesis, a diselenium-bond replaced the intra-molecular disulfide bond in the A chain. sCon-Ins-G1 induces hypoglycemic shock when it is injected into fish, and it slows fish motility when it is present in the water. Other than its effects on fish, the most special feature of Con-Ins-G1 is that it is the shortest insulin molecule reported to date with a “shortened” B chain. Because a shortened human insulin (des-octapeptide insulin, DOI) is monomeric, it indicated that Con-Ins-G1 is monomeric and can be used as an UFI. Con-Ins-G1 lacks two segments that in human insulin are involved in binding to with the human insulin receptor (hIR): First, A21 Asn of human insulin contacts hIR binding site 1 and its removal causes a 100-fold reduction in binding affinity. Second, the aromatic triplet (B24-B26) is one element for human insulin to bind hIR binding through contacts at hIR binding site 1. Removal of these residues leads to a 1,000-fold reduction in affinity.

Despite these concerns, Con-Ins-G1 (instead of the selenium analogue) was chemically synthesized and it was found that it binds to hIR with only 30-fold less affinity than human insulin. This surprising result raised a key question: how does Con-Ins-G1 bind to hIR without the key aromatic residues used by human insulin? The structure of Con-Ins-G1 was found to display a nearly identical backbone as human insulin. By fitting the Con-Ins-G1 structure into a published human insulin-hIR co-structure, it was inferred that Con-Ins-G1 B15 Tyr and B20 Tyr (Leu and Gly in human insulin) interact with human IR to substitute for the role played by human B24 Phe. These strong results provide a rational basis to develop a human monomeric UFI based on the snail insulin structure.

1. Develop Human Monomeric Insulin Analogs as Therapeutic Leads.

The development of ultra-fast acting insulin represents the next major advance in insulin analogue development. The fundamental challenge in redesigning human insulin is that the same residues involved in receptor binding also mediate dimer formation. Thus, the discovery of the venomous insulin Con-Ins-G1 represents an important step forward in the creation of a monomeric, ultrafast-acting insulin because it lacks these residues (and thus does not dimerize) but retains the ability to bind and activate the insulin receptor. There is concern, however, that the low sequence identity between Con-Ins-G1 and human insulin could give rise to an immune response, especially given that diabetes is a chronic disease that requires daily insulin injections. Therefore, instead of developing an UFI based on the venomous insulin, one can start with the scaffold of human DOI (Des-octapeptide (B23-30) human insulin) because it is monomeric and because close analogs of this truncated human insulin are likely to be tolerated by the human immune system, as indicated by the current clinical use of insulin analogues displaying two or three mutations. The challenge, however, is that DOI is nearly inactive (1,000-fold weaker than human insulin). Data indicate that Con-Ins-G1 uses the B15 Tyr and/or B20 Tyr to compensate for the loss of B24 Phe, and further indicate additional modifications that enhance the affinity of Con-Ins-G1. Leveraging these insights, DOI can be developed into an active UFI analogue as a therapeutic lead for diabetes treatment.

i. Develop Human DOI into a Bioactive Monomeric Insulin

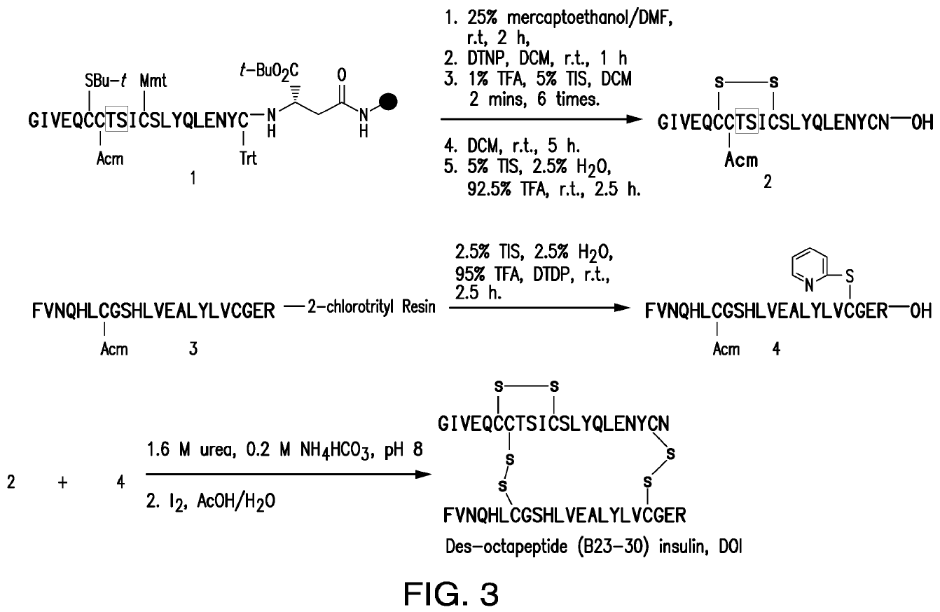

Traditionally, DOI was synthesized enzymatically by trypsin cleavage of human insulin, which is not suitable for analogue synthesis. Therefore, a modular synthetic route to access DOI has been developed. The primary challenge for the synthesis of human insulin is the hydrophobic character of the A chain. By using an isoacyl peptide pair on the A8-A9 Thr-Ser, an extra charged residue (amine) was introduced to the A chain to increase its solubility (FIG. 3). After disulfide bond formation, the isoacyl peptide underwent an O-to-N acyl shift at pH 8 to yield the DOI sequence. This synthetic DOI has the same molecular weight (from MALDI) and hIR activation activity as the enzymatically synthetic DOI, which proves the reliability of the developed method.

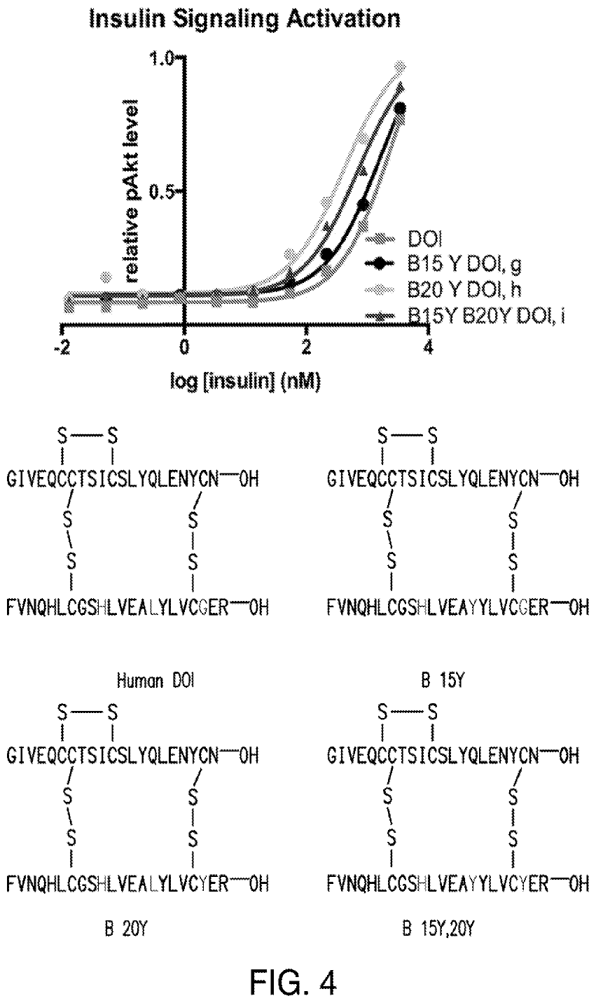

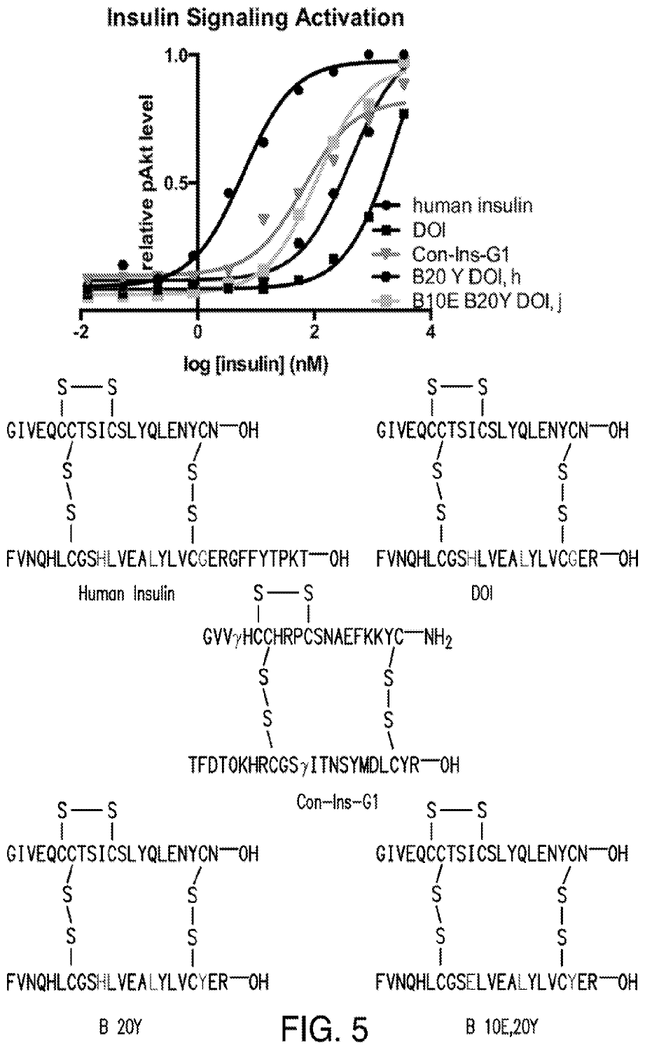

It has been demonstrated that the two Tyr on B15 and B20 of Con-Ins-G1 are important for hIR activation. To test the hypothesis that mutations on these two sites will increase the potency of hIR activation, three DOI analogues with B15 Leu and/or B20 Gly mutated to Tyr were synthesized. As shown in FIG. 4, the two analogues with B20 Tyr have 5-fold increased potency in hIR activation while the B15 Tyr DOI analogue is similar to DOI. This demonstrates that B20 Tyr alone can increase the potency of DOI, likely due to compensation for loss of B24 Phe. To further increase potency, a DOI analogue that additionally displays B10 Glu was synthesized, which is the B10 substitution that gives the strongest hIR binding. This provided another 5-fold increase in potency compared to B20 Tyr alone, and has a similar potency as Con-Ins-G1 (FIG. 5). This demonstrates that mutations from the venomous insulin can be grafted onto human DOI to develop bioactive analogues.

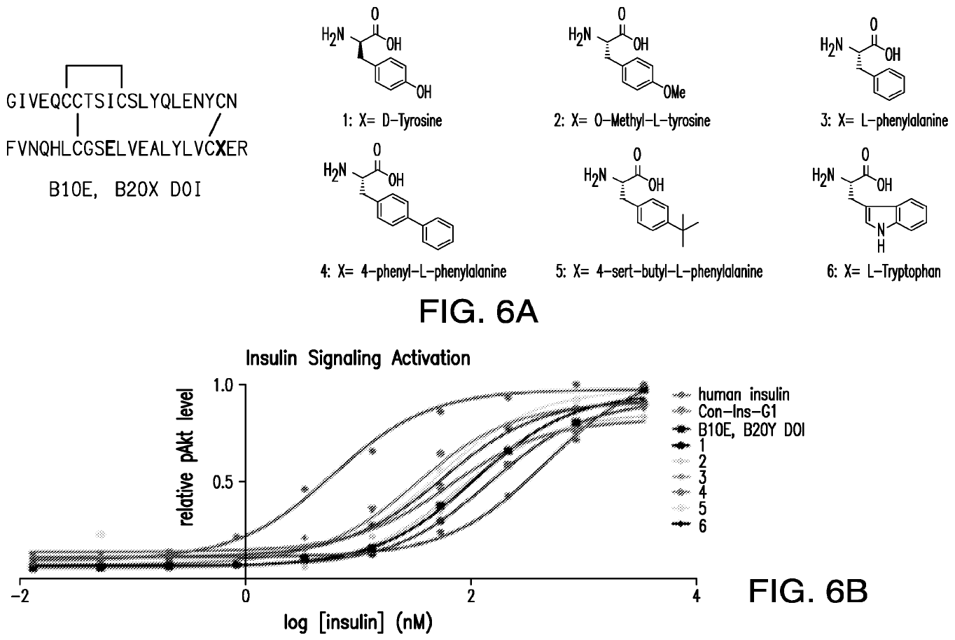

The crystal structure of Con-Ins-G1 lacks clear electron density for the B20 residue, which indicates that it may be flexible. Therefore, the hypothesis that substitutions other than Tyr can further increase potency were tested by synthesizing a series of B10E, B20X DOI analogues with X being aromatic amino acids (FIG. 6A). Interestingly, large substituents such as indole (Trp) and biphenyl group lead to higher potency in hIR activation (FIG. 6B). The biphenyl analogue is 10% of the potency of human insulin (3-fold higher than N10E, B20Y DOI). This demonstrates the power of the interdisciplinary approach using both protein engineering and structural biology. The potency of DOI has been increased by 100-fold by mutating two positions. Halogen-substituted naphthyl and biphenyl groups on B20 can be used to further optimize DOI analogue potency.

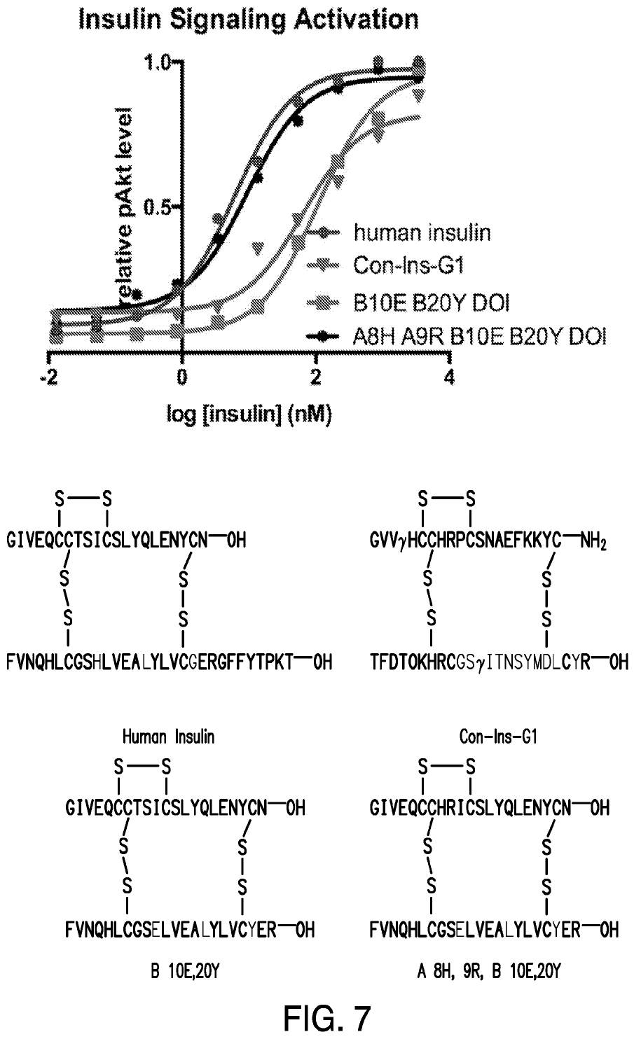

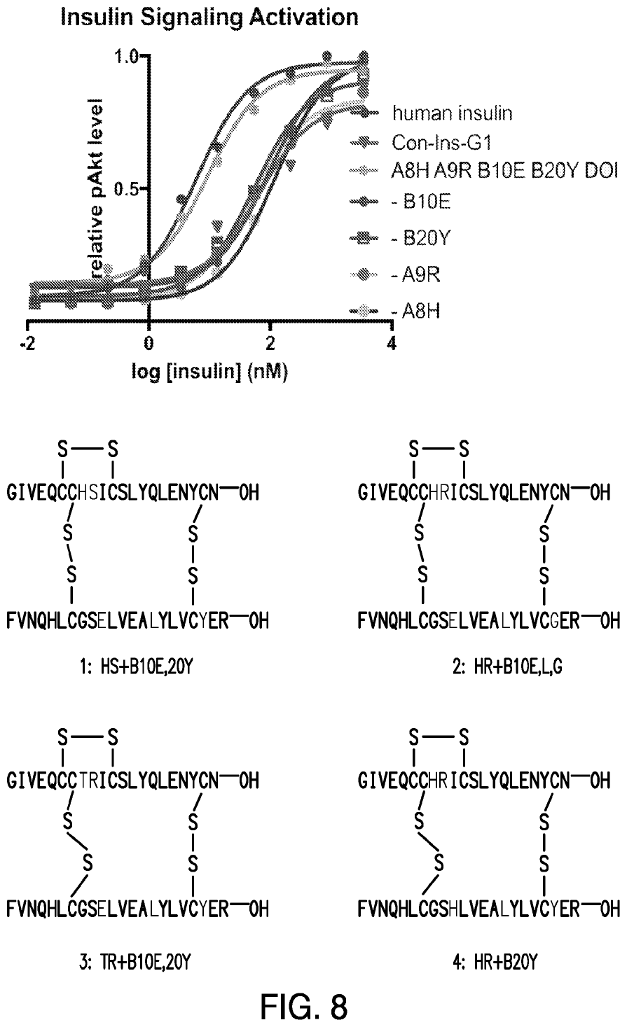

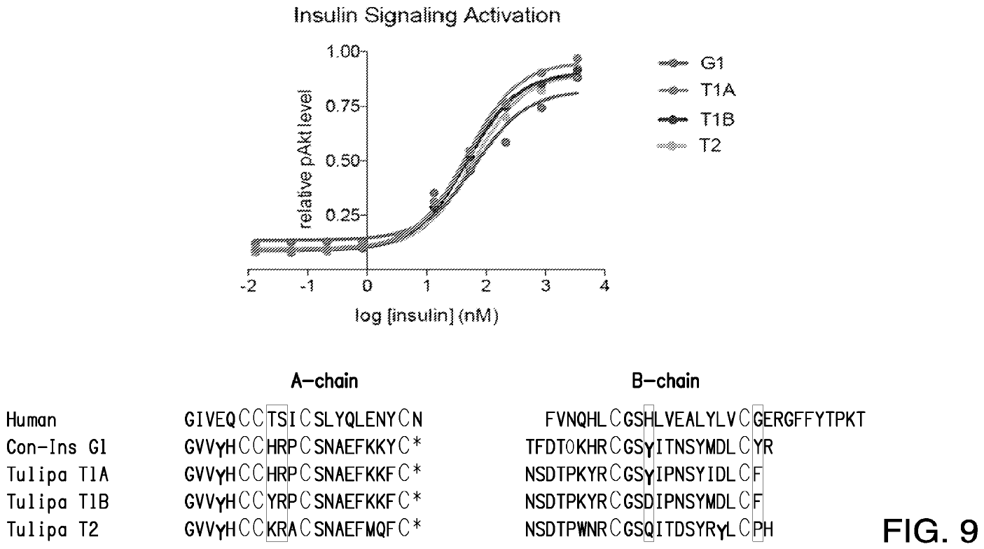

Because the A8 position is important for interacting with hIR binding site 2, the A8 His mutation can be introduced into the current lead analogue and assay for hIR activation. Both A8 His and A9 Arg (original residues on Con-Ins-G1) were introduced to the DOI analogue with B10 Glu and B20 Tyr, the lead analogue (FIG. 5). This quadruple DOI mutant has potency for hIR activation that is comparable to that of human insulin (FIG. 7). The mutations on Con-Ins-G1 promote binding to IR site 2. X-ray crystallography can be used to study the interaction between insulin and binding site 2. Protein engineering efforts can be expanded to the A8-A10 triplet to further optimize interaction with hIR binding site 2 by using a medicinal chemistry approach similar to the work on B20. Currently, the best analog varies from the parent human insulin sequence at only 4 residues, so it is likely that the immunogenicity of the monomeric DOI analogues will be similar to that of the FDA-approved insulin analogues that are in clinical use.

It was demonstrated that each mutation on A8, A9, B10 and B20 has individual effects in affect hIR activation (FIG. 8).

ii. Evaluate Monomeric Insulin Leads in STZ Treated Diabetic Mouse Models.

After potent monomeric insulin analogues are identified, the in vivo properties can be evaluated. An insulin tolerance test can be performed in STZ-treated mice to confirm the in vivo glucose-lowering ability. The two key features for an UFI analogue are fast onset and short duration of action. UFI analogue serum levels will be measured using HPLC coupled with mass spectrometry (LC/MS/MS) in diabetic mice after subcutaneous injections to measure its absorption rate (using insulin lispro as a control). For monomeric insulins, a faster absorption rate can be seen compared to the dimeric insulin lispro. Furthermore, glycemic clamp experiments can be used to quantify the onset and duration of UFI analogues in vivo by determining the amount of glucose infusion required to maintain a targeted glucose level. The glucose clamp study can show that UFI analogues have a shorter onset and duration of action due to their reduced depot effects in subcutaneous tissue. The combination of these properties can greatly reduce the risk of hypoglycemia.

Those skilled in the art will recognize, or be able to ascertain using no more than routine experimentation, many equivalents to the specific embodiments of the method and compositions described herein. Such equivalents are intended to be encompassed by the following claims.

1. A peptide comprising an insulin A chain peptide and an insulin B chain peptide, wherein the insulin A chain peptide comprises the amino acid sequence GIVEQCCTSICSLYQLENYCHALQ.

2. The peptide of claim 1, wherein the B chain peptide comprises a substitution at amino acid 10 and amino acid 20.

3. The peptide of claim 1, wherein the substitution at amino acid 20 is G02L, G20Y, G20F, or G20P.

4. The peptide of claim 1, wherein the substitution at amino acid 10 is H10E, H10D or H10Q.

5. The peptide of claim 1, further comprising at least one substitution in the A chain peptide, wherein the at least one substitution is not present in the HALQ sequence.

6. The peptide of claim 5, wherein the at least one substitution in the A chain peptide is T8H, T8Y, T8K, or S9R.

7. The peptide of claim 6, further comprising at least two substitutions in the A chain peptide, wherein the at least two substitutions are not present in the HALQ sequence.

8. The peptide of claim 5, wherein the at least two substitutions in the A chain peptide are two of the substitutions selected from: T8H, T8Y, T8K, and S9R.

9. The peptide of claim 1, wherein the peptide is a des-octapeptide insulin.

10. The peptide of any of claim 1, wherein the B chain peptide comprises the sequence of FVNQHLCGSELVEALYLVCYER (SEQ ID NO: 3).

11. The peptide of claim 1, wherein the A chain peptide and B chain peptide are bonded via at least one disulfide bond.

12. The peptide of claim 1, wherein the peptide is a monomer.

13. The peptide of claim 1, wherein the insulin A chain peptide is at least 70% identical to wild type human insulin A chain peptide.

14. A pharmaceutical composition comprising the peptide of claim 1 and a pharmaceutically acceptable carrier.

15. A method of increasing insulin receptor activation in a subject comprising administering a therapeutically effective amount of the peptide of claim 1 to a subject in need thereof.

16. A method of lowering the blood sugar in a subject comprising administering a therapeutically effective amount of the peptide of claim 1 to a subject in need thereof.

17. A method of treating type 1 diabetes in a subject comprising administering a therapeutically effective amount of the peptide of claim 1 to a subject in need thereof.

18. The method of claim 17, wherein the subject has been diagnosed with type 1 diabetes prior to administering the peptide.

19. A therapeutic protein having an A chain peptide bonded to a B chain peptide via at least one disulfide bond, wherein the A chain comprises the sequence of GIVEQCCTSICSLYQLENYCHALQ (SEQ ID NO: 31), and wherein the B chain peptide comprises the sequence of FVNQHLCGSELVEALYLVCLER (SEQ ID NO: 35).

20. The compositions and peptides disclosed herein.
